(19) United States
(12) Patent Application Publication
(10) Pub. No.: US 2022/0341947 A1
(43) Pub. Date: Oct. 27, 2022

(54) COMPOSITIONS AND METHODS FOR DIAGNOSIS OF PERIPHERAL ARTERIAL DISEASE

(71) Applicant: UNITY HEALTH TORONTO, Toronto (CA)

(72) Inventor: Mohammad QADURA, Toronto (CA)

(21) Appl. No.: 17/763,992

(22) PCT Filed: Sep. 25, 2020
(86) PCT No.: PCT/CA2020/051287

§ 371 Date: Mar. 25, 2022

Related U.S. Application Data
(60) Provisional application No. 62/906,984, filed on Sep. 27, 2019.

(51) Int. Cl. G01N 33/68; G01N 33/92; C07K 14/47

(52) U.S. Cl. CPC G01N 33/6893; G01N 33/92; C07K 14/47; G01N 2800/56; G01N 2800/52

(57) ABSTRACT

Described herein is fatty acid-binding protein 3 (FABP3) and/or FABP4 for diagnosing or staging peripheral artery disease (PAD) or for assessing revascularization in PAD afflicted subjects. Also described is FABP3 and/or FABP4 for distinguishing PAD patients from non-PAD patients regardless of the presence PAD symptoms, for distinguishing PAD patients with a non-compressible ABI from non-PAD patients, or for determining prognosis in PAD. Also described herein are various methods and biomarker panels for use in PAD.

### FIELD

The present invention relates to peripheral arterial disease (PAD). In particular, the present invention relates to compositions and methods for diagnosis of peripheral arterial disease.

### BACKGROUND

Fatty acid-binding protein 3 (FABP3), also known as heart type fatty acid-binding protein (hFABP), is a small cytoplasmic protein that is thought to participate in the intracellular trafficking and metabolism of long-chain fatty acids. Fatty acid-binding protein 4 (FABP4), also known as adipocyte Protein 2 (aP2) is a carrier protein for fatty acids that is primarily expressed in adipocytes and macrophages. Peripheral artery disease (PAD) is an abnormal narrowing of arteries other than those that supply the heart or brain.

U.S. Pat. No. 8,062,857 describes a method for diagnosing myocardial infarction in a subject based on the determination of H-FABP and, optionally, myoglobin in a sample of the subject. U.S. Pat. No. 7,754,436 describes a diagnostic assay for H-FABP or B-FABP that distinguishes between stroke and acute myocardial infarction. U.S. Patent Application Publication No. 2017/0219608 describes a kit for testing myocardial infarction comprising a strip capable of detecting three markers, namely, human myeloperoxidase (MPO), heart-fatty acid binding protein (FABP3) and cardiac troponin I (cTnI) simultaneously. Karbek et al. (Cardiovascular Diabetology 2011, 10:37) describe that serum H-FABP levels could represent a useful marker for myocardial performance in patients with diabetes. Otaki et al. (BBA Clinical 4 (2015) 35-41) describe that the myocardial damage markers H-FABP and hsTnT were increased in PAD patients with CLI and could predict MACCEs in PAD patients. Pritt et al. describe FABP3 as a biomarker of skeletal muscle toxicity in the rat.

Despite this, there is a need to develop biomarkers and methods associated with PAD.

### SUMMARY OF THE INVENTION

In accordance with an aspect, there is provided fatty acid-binding protein 3 (FABP3) and/or FABP4 for diagnosing peripheral artery disease (PAD).

In accordance with an aspect, there is provided fatty acid-binding protein 3 (FABP3) and/or FABP4 for staging peripheral artery disease (PAD).

In accordance with an aspect, there is provided fatty acid-binding protein 3 (FABP3) and/or for assessing revascularization status in a subject afflicted with peripheral artery disease (PAD).

In accordance with an aspect, there is provided fatty acid-binding protein 3 (FABP3) and/or FABP4 distinguishing peripheral artery disease (PAD) patients from non-PAD patients regardless of the presence PAD symptoms.

In accordance with an aspect, there is provided fatty acid-binding protein 3 (FABP3) and/or FABP4 for distinguishing peripheral artery disease (PAD) patients with a non-compressible ABI from non-PAD patients.

In accordance with an aspect, there is provided fatty acid-binding protein 3 (FABP3) and/or FABP4 for determining prognosis in peripheral artery disease (PAD).

In an aspect, the FABP3 and/or FABP4 is in combination with at least one other biomarker.

In an aspect, the at least one other biomarker comprises high sensitivity troponin, troponin I (TnI), troponin T (TnT), FABP3, FABP4, or a combination thereof.

In accordance with an aspect, there is provided a panel of biomarkers for assessing peripheral artery disease (PAD), the panel comprising FABP3 and/or FABP4 and at least one additional biomarker.

In an aspect, the at least one additional biomarker comprises a biomarker associated with PAD.

In an aspect, the at least one additional biomarker comprises a biomarker associated with myocardial ischemia.

In an aspect, the biomarker associated with myocardial ischemia is high sensitivity troponin, troponin I (TnI), and/or troponin T (TnT).

In an aspect, the at least one additional biomarker comprises the other of FABP3 and/or FABP4.

In an aspect, a detected level of FABP3 protein in a patient sample of <0.6 ng/ml, <0.7 ng/ml, <0.8 ng/ml, <0.9 ng/ml, <1.0 ng/ml, <1.1 ng/ml, <1.2 ng/ml, <1.3 ng/ml, <1.4 ng/ml, <1.5 ng/ml, <1.6 ng/ml, <1.7 ng/ml, <1.8 ng/ml, <1.9 ng/ml, <2.0 ng/ml, <2.1 ng/ml, or <2.2 ng/ml is suggestive that the subject is highly unlikely to have PAD.

In an aspect, a detected level of FABP3 protein in a patient sample of ≥0.6 ng/ml and <4.5 ng/ml, such as ≥0.6 ng/ml, ≥0.7 ng/ml, ≥0.8 ng/ml, ≥0.9 ng/ml, ≥1.0 ng/ml, ≥1.1 ng/ml, ≥1.2 ng/ml, ≥1.3 ng/ml, ≥1.4 ng/ml, ≥1.5 ng/ml, ≥1.6 ng/ml, ≥1.7 ng/ml, ≥1.8 ng/ml, ≥1.9 ng/ml, ≥2.0 ng/ml, ≥2.1 ng/ml, or ≥2.2 ng/ml, and <3.5 ng/ml, <3.6 ng/ml, <3.7 ng/ml, <3.8 ng/ml, <3.9 ng/ml, <4.0 ng/ml, <4.1 ng/ml, <4.2 ng/ml, <4.3 ng/ml, <4.4 ng/ml, and <4.5 ng/ml is suggestive that the subject is at moderate risk of having PAD.

In an aspect, a detected level of FABP3 protein in a patient sample of ≥3.5 ng/ml and <5.3 ng/ml, such as ≥3.5 ng/ml, ≥3.6 ng/ml, ≥3.7 ng/ml, ≥3.8 ng/ml, ≥3.9 ng/ml, ≥4.0 ng/ml, ≥4.1 ng/ml, ≥4.2 ng/ml, ≥4.3 ng/ml, ≥4.4 ng/ml, or ≥4.3 ng/ml, and <4.4 ng/ml, <4.5 ng/ml, <4.6 ng/ml, <4.7 ng/ml, <4.8 ng/ml, <4.9 ng/ml, <5.0 ng/ml, <5.1 ng/ml, <5.2 ng/ml, and <5.3 ng/ml is suggestive that the subject is at moderate-high risk of having PAD.

In an aspect, a detected level of FABP3 protein in a patient sample of ≥4.6 ng/ml, ≥4.7 ng/ml, ≥4.8 ng/ml, ≥4.9 ng/ml, ≥5.0 ng/ml, ≥5.1 ng/ml, ≥5.2 ng/ml, or ≥5.3 ng/ml is suggestive that the subject is at high risk of having PAD.

In an aspect, a detected level of FABP4 protein in a patient sample of <15 ng/ml, <16 ng/ml, <17 ng/ml, <18 ng/ml, <19 ng/ml, <20 ng/ml, <21 ng/ml, <22 ng/ml, <23 ng/ml, <24 ng/ml, or <25 ng/ml is suggestive that the subject has PAD.

In accordance with an aspect, there is provided an assay comprising the FABP3 and/or FABP4 or the panel described herein.

In an aspect, the assay is a point of care assay.

In accordance with an aspect, there is provided a kit comprising the FABP3 and/or FABP4 or the panel described herein.

In accordance with an aspect, there is provided a method for diagnosing peripheral artery disease (PAD) in a subject, the method comprising detecting the level of fatty acid-binding protein 3 (FABP3) and/or FABP4 in the subject; wherein an elevated level of FABP3 and/or FABP4 is indicative of PAD in the subject.

In an aspect, the elevated level of FABP3 and/or FABP4 in the subject is determined by comparing the detected level of FABP3 and/or FABP4 to a control level of FABP3 and/or FABP4.

In an aspect, the control level of FABP3 and/or FABP4 is a predetermined value obtained from one or a pool of non-PAD patients or healthy patients.

In an aspect, the method further comprises detecting the level of at least one additional biomarker.

In an aspect, the at least one additional biomarker comprises the other of FABP3 and/or FABP4, high sensitivity troponin, TnI, TnT, and/or creatinine.

In an aspect, the method further comprises assessing the ABI of the subject.

In an aspect, the PAD is non-symptomatic (stage 0), mild PAD (stage 1), moderate PAD (stage 2), severe PAD (stage 3), early chronically threatened limb ischemia (CTLI) (stage 4) or advanced CTLI (stages 5-6).

In an aspect, the PAD is early or advanced CTLI.

In an aspect, the subject is free of clinical and/or biochemical evidence of myocardial ischemia.

In an aspect, the method further comprises detecting the level of high sensitivity troponin, troponin I (TnI) and/or troponin T (TnT) in the subject, wherein a substantially normal level of high sensitivity troponin, TnI and/or TnT in the subject is further indicative of PAD in the subject.

In an aspect, the substantially normal level of high sensitivity troponin, TnI and/or TnT in the subject is determined by comparing the detected level of high sensitivity troponin, TnI and/or TnT to a control level of TnI and/or TnT.

In an aspect, the subject is free of clinical and/or biochemical evidence of kidney dysfunction.

In an aspect, the method further comprises detecting the level of creatinine in the subject, wherein a substantially normal level of creatinine in the subject is further indicative of PAD in the subject.

In an aspect, the substantially normal level of creatinine in the subject is determined by comparing the detected level of creatinine to a control level of creatinine.

In an aspect, the subject is free of clinical and/or biochemical evidence of acute stroke and/or acute muscle toxicity.

In an aspect, the subject has a concurrent condition and optionally wherein the detected level of FABP3 and/or FABP4 and/or the control level of FABP3 and/or FABP4 is optionally adjusted for the concurrent condition.

In an aspect, the concurrent condition is kidney dysfunction, stroke, diabetes, and/or muscle toxicity.

In accordance with an aspect, there is provided a method for staging peripheral artery disease (PAD) in a subject, the method comprising detecting the level of fatty acid-binding protein 3 (FABP3) and/or FABP4 in the subject; wherein an elevated level of FABP3 correlates with the stage of PAD in the subject.

In an aspect, the elevated level of FABP3 and/or FABP4 in the subject is determined by comparing the detected level of FABP3 and/or FABP4 to a control level of FABP3 and/or FABP4, and wherein the size of the difference between the detected level of FABP3 and/or FABP4 and the control level of FABP3 positively correlates with the stage of PAD in the subject.

In an aspect, the control level of FABP3 and/or FABP4 is a predetermined value obtained from one or a pool of non-PAD patients or healthy patients.

In an aspect, the method further comprises detecting the level of at least one additional biomarker.

In an aspect, the at least one additional biomarker comprises the other of FABP3 and/or FABP4, high sensitivity troponin, TnI, TnT, and/or creatinine.

In an aspect, the method further comprises assessing the ABI of the subject.

In an aspect, the method comprises staging the PAD as asymptomatic (stage 0), mild PAD (stage 1), moderate PAD (stage 2), severe PAD (stage 3), early CTLI (stage 4) or late CTLI (stage 5-6) based on the detected level of FABP3.

In an aspect, the subject is free of clinical and/or biochemical evidence of myocardial ischemia.

In an aspect, the method further comprises detecting the level of high sensitivity troponin, troponin I (TnI) and/or troponin T (TnT) in the subject, wherein a substantially normal level of high sensitivity troponin, TnI and/or TnT in the subject is further indicative of PAD in the subject.

In an aspect, the substantially normal level of high sensitivity troponin, TnI and/or TnT in the subject is determined by comparing the detected level of high sensitivity troponin, TnI and/or TnT to a control level of high sensitivity troponin, TnI and/or TnT.

In an aspect, the subject is free of clinical and/or biochemical evidence of kidney dysfunction.

In an aspect, the method further comprises detecting the level of creatinine in the subject, wherein a substantially normal level of creatinine in the subject is further indicative of PAD in the subject.

In an aspect, the substantially normal level of creatinine in the subject is determined by comparing the detected level of creatinine to a control level of creatinine.

In an aspect, the subject is free of clinical and/or biochemical evidence of acute stroke and/or muscle toxicity.

In an aspect, the subject has a concurrent condition and wherein the detected level of FABP3 and/or FABP4 and/or the control level of FABP3 and/or FABP4 is optionally adjusted for the concurrent condition.

In an aspect, the concurrent condition is kidney dysfunction, stroke, diabetes, and/or muscle toxicity.

In accordance with an aspect, there is provided a method for assessing revascularization in a subject with peripheral artery disease (PAD), the method comprising detecting the level of fatty acid-binding protein 3 (FABP3) and/or FABP4 in the subject; wherein a substantially normal level of FABP3 and/or FABP4 or a reduction in an elevated level of FABP3 and/or FABP4 is indicative of arterial revascularization in the subject.

In an aspect, the substantially normal level of FABP3 and/or FABP4 or the reduction in the elevated level of FABP3 and/or FABP4 is determined by comparing the detected level of FABP3 and/or FABP4 to a control level of FABP3 and/or FABP4.

In an aspect, the control level of FABP3 and/or FABP4 is a predetermined value obtained from one or a pool of non-PAD patients or healthy patients.

In an aspect, the control level of FABP3 and/or FABP4 is a predetermined value obtained from one or a pool of PAD patients.

In an aspect, the control level of FABP3 and/or FABP4 is the level of FABP3 and/or FABP4 detected in the subject prior to revascularization treatment.

In an aspect, the method further comprises detecting the level of at least one additional biomarker.

In an aspect, the at least one additional biomarker comprises the other of FABP3 and/or FABP4, high sensitivity troponin, TnI, TnT, and/or creatinine.

In an aspect, the method further comprises assessing the ABI of the subject.

In an aspect, the PAD is asymptomatic (stage 0), mild PAD (stage 1), moderate PAD (stage 2), severe PAD (stage 3), early CTLI (stage 4) or advanced CTLI (stages 5-6).

In an aspect, the PAD is early or advanced CTLI.

In an aspect, the subject is free of clinical and/or biochemical evidence of myocardial ischemia.

In an aspect, the method further comprises detecting the level of high sensitivity troponin, troponin I (TnI) and/or troponin T (TnT) in the subject, wherein a substantially normal level of high sensitivity troponin, TnI and/or TnT in the subject is further indicative of revascularization in the subject.

In an aspect, the substantially normal level of high sensitivity troponin, TnI and/or TnT in the subject is determined by comparing the detected level of high sensitivity troponin, TnI and/or TnT to a control level of high sensitivity troponin, TnI and/or Tnt.

In an aspect, the subject is free of clinical and/or biochemical evidence of kidney dysfunction.

In an aspect, the method further comprises detecting the level of creatinine in the subject, wherein a substantially normal level of creatinine in the subject is further indicative of revascularization in the subject.

In an aspect, the substantially normal level of creatinine in the subject is determined by comparing the detected level of creatinine to a control level of creatinine.

In an aspect, the subject is free of clinical and/or biochemical evidence of acute stroke and/or muscle toxicity.

In an aspect, the subject has a concurrent condition and wherein the detected level of FABP3 and/or FABP4 and/or the control level of FABP3 and/or FABP4 is optionally adjusted for the concurrent condition.

In an aspect, the concurrent condition is kidney dysfunction, stroke, diabetes, and/or muscle toxicity.

In accordance with an aspect, there is provided a method for predicting whether a subject with peripheral artery disease (PAD) is likely to progress to CTLI, the method comprising detecting the level of fatty acid-binding protein 3 (FABP3) and/or FABP4 in the subject; wherein the extent of elevation of FABP3 and/or FABP4 is correlated with the likelihood of the subject progressing to CTLI.

In an aspect, the extent of elevation of FABP3 and/or FABP4 in the subject is determined by comparing the detected level of FABP3 and/or FABP4 to a control level of FABP3 and/or FABP4.

In an aspect, the control level of FABP3 and/or FABP4 is a predetermined value obtained from one or a pool of non-PAD patients or healthy patients.

In an aspect, the method further comprises detecting the level of at least one additional biomarker.

In an aspect, the at least one additional biomarker comprises the other of FABP3 and/or FABP4, high sensitivity troponin, TnI, TnT, and/or creatinine.

In an aspect, the method further comprises assessing the ABI of the subject.

In an aspect, the subject is free of clinical and/or biochemical evidence of myocardial ischemia.

In an aspect, the method further comprises detecting the level of high sensitivity troponin, troponin I (TnI) and/or troponin T (TnT) in the subject, wherein a substantially normal level of high sensitivity troponin, TnI and/or TnT in the subject is further indicative of PAD in the subject.

In an aspect, the substantially normal level of high sensitivity troponin, TnI and/or TnT in the subject is determined by comparing the detected level of high sensitivity troponin, TnI and/or TnT to a control level of high sensitivity troponin, TnI and/or TnT.

In an aspect, the subject is free of clinical and/or biochemical evidence of kidney dysfunction.

In an aspect, the method further comprises detecting the level of creatinine in the subject, wherein a substantially normal level of creatinine in the subject is further indicative of PAD in the subject.

In an aspect, the substantially normal level of creatinine in the subject is determined by comparing the detected level of creatinine to a control level of creatinine.

In an aspect, the subject is free of clinical and/or biochemical evidence of acute stroke and/or muscle toxicity.

In an aspect, the subject has a concurrent condition and wherein the detected level of FABP3 and/or FABP4 and/or the control level of FABP3 and/or FABP4 is optionally adjusted for the concurrent condition.

In an aspect, the concurrent condition is kidney dysfunction, stroke, diabetes and/or muscle toxicity.

In an aspect, the FABP3 and/or FABP4 is detected in whole blood, plasma, urine, saliva, oral fluid, cerebrospinal fluid, amniotic fluid, milk, colostrum, mammary gland secretion, lymph, sweat, lacrimal fluid, gastric fluid, synovial fluid, mucus, or combinations thereof.

In an aspect, the FABP3 and/or FABP4 is detected as protein, DNA, RNA, or a combination thereof.

In an aspect, the subject is an adult.

In an aspect, the subject is at least 20, 25, 30, 35, 40, 45, 50, 55, 60, 65, 70, 75, or 80 years of age.

In an aspect, a detected level of FABP3 and/or FABP4 protein in a patient sample of <0.6 ng/ml, <0.7 ng/ml, <0.8 ng/ml, <0.9 ng/ml, <1.0 ng/ml, <1.1 ng/ml, <1.2 ng/ml, <1.3 ng/ml, <1.4 ng/ml, <1.5 ng/ml, <1.6 ng/ml, <1.7 ng/ml, <1.8 ng/ml, <1.9 ng/ml, <2.0 ng/ml, <2.1 ng/ml, or <2.2 ng/ml is suggestive that the subject is highly unlikely to have PAD.

In an aspect, a detected level of FABP3 and/or FABP4 protein in a patient sample of ≥0.6 ng/ml and <4.5 ng/ml, such as ≥0.6 ng/ml, ≥0.7 ng/ml, ≥0.8 ng/ml, ≥0.9 ng/ml, ≥1.0 ng/ml, ≥1.1 ng/ml, ≥1.2 ng/ml, ≥1.3 ng/ml, ≥1.4 ng/ml, ≥1.5 ng/ml, ≥1.6 ng/ml, ≥1.7 ng/ml, ≥1.8 ng/ml, ≥1.9 ng/ml, ≥2.0 ng/ml, ≥2.1 ng/ml, or ≥2.2 ng/ml, and <3.5 ng/ml, <3.6 ng/ml, <3.7 ng/ml, <3.8 ng/ml, <3.9 ng/ml, <4.0 ng/ml, <4.1 ng/ml, <4.2 ng/ml, <4.3 ng/ml, <4.4 ng/ml, and <4.5 ng/ml is suggestive that the subject is at moderate risk of having PAD.

In an aspect, a detected level of FABP3 and/or FABP4 protein in a patient sample of ≥3.5 ng/ml and <5.3 ng/ml, such as ≥3.5 ng/ml, ≥3.6 ng/ml, ≥3.7 ng/ml, ≥3.8 ng/ml, ≥3.9 ng/ml, ≥4.0 ng/ml, ≥4.1 ng/ml, ≥4.2 ng/ml, ≥4.3 ng/ml, ≥4.4 ng/ml, or ≥4.3 ng/ml, and <4.4 ng/ml, <4.5 ng/ml, <4.6 ng/ml, <4.7 ng/ml, <4.8 ng/ml, <4.9 ng/ml, <5.0 ng/ml, <5.1 ng/ml, <5.2 ng/ml, and <5.3 ng/ml is suggestive that the subject is at moderate-high risk of having PAD.

In an aspect, a detected level of FABP3 and/or FABP4 protein in a patient sample of ≥4.6 ng/ml, ≥4.7 ng/ml, ≥4.8 ng/ml, ≥4.9 ng/ml, ≥5.0 ng/ml, ≥5.1 ng/ml, ≥5.2 ng/ml, or ≥5.3 ng/ml is suggestive that the subject is at high risk of having PAD.

In an aspect, a detected level of FABP4 protein in a patient sample of <15 ng/ml, <16 ng/ml, <17 ng/ml, <18 ng/ml, <19 ng/ml, <20 ng/ml, <21 ng/ml, <22 ng/ml, <23 ng/ml, <24 ng/ml, or <25 ng/ml is suggestive that the subject has PAD.

In an aspect, the method further comprising treating the subject based upon the outcome of the method.

In accordance with an aspect, there is provided a method of treating a subject with peripheral artery disease, the method comprising carrying out at least one method described herein and treating the subject based upon the outcome of the method.

In accordance with an aspect, there is provided a use of FABP3 and/or FABP4 for diagnosing peripheral artery disease (PAD) in a subject, wherein an elevated level of FABP3 and/or FABP4 is indicative of PAD in the subject.

In accordance with an aspect, there is provided a use of FABP3 and/or FABP4 for staging peripheral artery disease (PAD) in a subject, wherein an elevated level of FABP3 and/or FABP4 correlates with the stage of PAD in the subject.

In accordance with an aspect, there is provided a use of FABP3 and/or FABP4 for assessing revascularization in a subject with peripheral artery disease (PAD); wherein a substantially normal level of FABP3 and/or FABP4 or a reduction in an elevated level of FABP3 is indicative of arterial revascularization in the subject.

In accordance with an aspect, there is provided a use of FABP3 and/or FABP4 for predicting whether a subject with peripheral artery disease (PAD) is likely to progress to CTLI, wherein the extent of elevation of FABP3 and/or FABP4 is correlated with the likelihood of the subject progressing to CTLI.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1. An algorithm to diagnose PAD based on the Plasma levels of FABP3. The exemplary cut-off points were established using 486 patients.

FIG. 2. Receiver Operating Characteristics (ROC) analyses on for FABP3 in plasma in 486 patients. Unadjusted ROC analysis for FABP3 in plasma samples obtained from 105 non-PAD controls and 381 PAD patients with an area under curve (AUC) of 0.8234 (95% CI, 0.7818 to 0.8651) is represented by the solid line. When compared to non-PAD control, our ROC analysis confirms the use of FABP3 in plasma as an excellent biomarker of PAD with large area under curve.

FIG. 3. Receiver Operating Characteristics (ROC) analyses for FABP3 in plasma of patients with CTLI. Unadjusted ROC analysis for FABP3 AUC 0.80 (95% CI, 0.65-0.87) is represented by the dotted line. The ROC analysis for FABP3 after adjusting for confounding factors is represented by the dashed line. The AUC for FABP3 improved to 0.92 (95% CI, 0.79-0.97) after adjusting for confounding factors.

FIG. 4. FABP3 levels positively correlated with the severity of PAD. A bar chart representing mean values with confidence intervals for FABP3 levels in 250 controls and PAD patients. Levels of FABP3 demonstrated an increase in FABP3 levels as the Rutherford classification increase. The (*) denotes statistical difference with P value <0.05 between the experimental group and control cohort.

FIG. 5. Levels of Troponin I (TnI) in healthy, Non-PAD, CTLI and acute coronary syndrome (ACS) patients. The level of TnI was measured in a large cohort of PAD, CTLI, ACS and control patients. 50 CTLI patients were matched to 25 non-PAD patients and 15 ACS patients. 15 healthy patients without risk factors were used as a negative control. Relative to healthy and non-PAD patients, increased levels of troponin I was only observed in the ACS group, and not in the CTLI group. The (*) denotes statistical difference with P value≤0.05.

FIG. 6. Increased FABP3 expression levels in the skeletal muscles of CTLI patients in comparison to non-PAD controls. A) Western blot demonstrating FABP3 and GAPDH expressions in the skeletal muscle of non-PAD controls (n=3) and CTLI patients (n=4). B) The histogram shows a quantitative representation of the levels of protein obtained from a densitometry analysis of four independent experiments. Each value represents the mean±standard error of the mean. A significant difference of comparison was determined by t-test as indicated by asterisk (*). P-value<0.05 versus control skeletal muscles. AU=absolute units.

FIG. 7. Immunohistochemistry (10×) of muscles obtained from non-PAD and CTLI patients. Hematoxylin-eosin (A,B), CD68 (C,D), Masson's Trichrome staining (E,F) and FABP3 (G.H) were used to assess cellular histology, Macrophages, muscular pathology/fibrosis, and localization of FABP3 in non-PAD and CTLI patients, respectively. This figure demonstrates that skeletal muscles is a source of expression of FABP3 in CTLI patients.

FIG. 8. Receiver Operating Characteristics (ROC) analyses for FABP3 in urine. Unadjusted ROC analysis for FABP3 in urine samples obtained from 41 non-PAD controls and 101 PAD patients with an area under curve (AUC) of 0.8644 (95% CI, 0.7939 to 0.9348) is represented by the solid line.

FIG. 9. Box plots representing FABP3 values across study groups categorized by symptoms. The levels of FABP3 are statistically increased in patients with asymptomatic PAD, symptomatic PAD and PAD patients with non-compressible (NS) ABI. * demonstrate a p-value of <0.001 between the experimental group and non-PAD controls.

FIG. 10. Box-whisker plots illustrating levels of FABP3 among PAD and non-PAD patients with and without confounding factors. FABP3 levels were higher in patients with PAD relative to controls regardless of sex; hypertension; hypercholesteremia; diabetes; smoking; age; coronary artery disease. Median FABP3 levels labeled in plots. Significant differences were calculated using Mann-Whitney test.

FIG. 11. Relationship between FABP3 and PAD. Spearman correlation between Ankle-Brachial-Index (ABI) and FABP3 in matched based on (Age, sex, Hypertension, Hypercholesteremia, Diabetes, Smoking, CAD) non-PAD (n=80, circle) and PAD (n=80, square). FABP3 values were inversely correlated to the ABI using spearmen correlation (r=−0.55, p-value=0.001).

FIG. 12. Line chart for the relation between FABP3 and walking distance. Correlation coefficient—0.39; p-value=0.001. This figure demonstrates the relationship between walking distance “clinical severity of PAD” and FABP3. As the walking distance is shortened “clinically worse PAD status”, elevated levels of FABP3 are noted.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed.

These terms of degree should be construed as including a deviation of at least ±5% of the modified term if this deviation would not negate the meaning of the word it modifies.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

As used herein, the term “biomarker” is intended to encompass a substance that is used as an indicator of a biologic state and includes genes (and nucleotide sequences of such genes), mRNAs (and nucleotide sequences of such mRNAs) and proteins (and amino acid sequences of such proteins). A “biomarker panel” includes a plurality of biomarkers, the expression of each of which is measured in order to provide a quantitative or qualitative summary of the expression of one or more biomarkers in a subject, such as in comparison to a standard or a control.

The terms “increased” or “increased expression” and “decreased” or “decreased expression”, with respect to the expression pattern of a biomarker(s), are used herein as meaning that the level of expression is increased or decreased relative to a constant basal level of expression of a household, or housekeeping, protein, whose expression level does not significantly vary under different conditions. A nonlimiting example of such a household, or housekeeping, protein is GAPDH. Other suitable household, or housekeeping, proteins are well-established in the art. In other aspects, these terms refer to an increase or decrease in the level of expression as compared to that observed in a control population, such as a subject or pool of subjects who have not experienced recent limb ischemia. In more typical aspects, these terms refer to an increase or decrease in relative concentrations in relation to the mean values of the sample in question.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human. In specific aspects, the biomarkers and methods described herein can be used in non-human animals. It will be understood that the biomarkers may not be completely conserved between the human versions described herein and equivalent animal versions, however, given the descriptions and examples provided here in it is understood that a skilled person could modify the biomarkers to be suitable for a desired animal population.

“Peripheral artery disease” (PAD) is an abnormal narrowing of arteries other than those that supply the heart or brain. Peripheral artery disease most commonly affects the legs, but other arteries may also be involved. Many patients with PAD are asymptomatic; the classic symptom PAD patients usually experience is calf pain while walking known as intermittent claudication. This pain resolves with rest. Other symptoms of advanced PAD include skin ulcers, bluish skin, cold skin, or abnormal nail and hair growth in the affected leg. Up to 50% of people with PAD do not have symptoms.

“Chronic limb threatening ischemia” (CLTI), also known as critical limb ischemia (CLI), is an advanced stage of peripheral artery disease (PAD). Compared earlier stages of PAD involving intermittent claudication, CTLI has a negative prognosis within a year after the initial diagnosis, with 1-year amputation rates of approximately 12% and mortality of 50% at 5 years and 70% at 10 years.

### Biomarkers and Panels

Fatty acid-binding protein 3 (FABP3), also known as heart type fatty acid-binding protein (hFABP), is a small cytoplasmic protein that is thought to participate in the intracellular trafficking and metabolism of long-chain fatty acids. Fatty acid-binding protein 3 (FABP4), also known as adipocyte Protein 2 (aP2) is a carrier protein for fatty acids that is primarily expressed in adipocytes and macrophages. Described herein is evidence that FABP3 is released from skeletal muscle tissue and is found in elevated levels in skeletal muscle, blood, and urine in subjects suffering from PAD. Similar findings with respect to FABP4 are also shown herein.

Thus, described herein is FABP3 and/or FABP4 for diagnosing or staging PAD. Further described herein is FABP3 and/or FABP4 for assessing revascularization status in subject afflicted with PAD. FABP3 and/or FABP4 may be used alone as a biomarker associated with PAD or it may be combined with other PAD biomarkers, such as the other of FABP3 and/or FABP4.

FABP3 may also be combined with a biomarker associated with myocardial ischemia, such as troponin, for example, high sensitivity troponin, troponin I (TnI) and/or troponin T (TnT). In this way, it can be determined whether an elevation in FABP3 and/or FABP4 is due to myocardial ischemia or PAD. For example, if FABP3 and/or FABP4 is elevated and troponin is within normal range, then it can be concluded that the FABP3 and/or FABP4 is likely elevated due to PAD and not myocardial ischemia. If, on the other hand, FABP3 and/or FABP4 and troponin are both elevated, then it is likely that the patient has more than one possible source of FABP3 and/or FABP4 release and more testing may be desired in order to determine if PAD is also present.

The biomarkers described herein may be assessed independently of one another or they may be assessed collectively in a panel. For example, a single blood or urine sample, for example, may be assessed in a single test to determine the levels of FABP3 together with FABP4 and/or high sensitivity troponin, and/or TnI and/or TnT, or any other desired biomarkers or combinations thereof.

In order to determine whether any given biomarker measurement is in a normal or elevated range, typically a cut-off or control level is used. The control levels may be, for example, based on one or a pool of healthy subjects not known to be afflicted by PAD or other pathologies such as myocardial ischemia. Alternatively or additionally, the control levels may be, for example, based on one or a pool of subjects afflicted with PAD or a specific stage of PAD but not myocardial ischemia. Alternatively or additionally, the control levels may be, for example, based on one or a pool of subjects afflicted with both PAD and myocardial ischemia. Combinations of these controls may be used in order to determine suitable ranges for comparison between detected levels of biomarkers and a given disease state or stage.

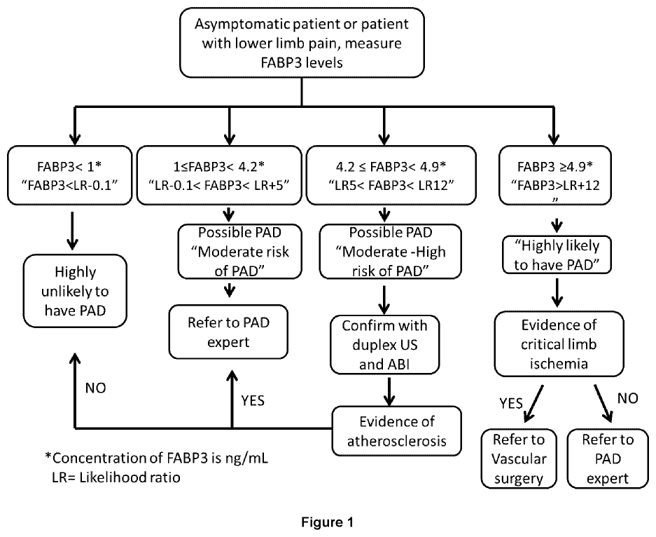

As an example, certain exemplary cut-offs are shown in FIG. 1. In this example, if FABP3 is measured as being less than 1.0 ng/ml, the subject is determined to be highly unlikely to have PAD. If FABP3 is measured as being between 1.0 ng/ml and 4.2 ng/ml, the subject is determined to have a moderate risk of PAD. If FABP3 is measured as being between 4.2 ng/ml and 4.9 ng/ml, the subject is determined to have a moderate-high risk of PAD. Finally, if FABP3 is measured as being higher than 4.9 ng/ml, the subject is determined to be highly likely to have PAD. As noted above, these cut-offs can be combined with other biomarker measurements and cut-offs to rule out myocardial ischemia as the source of FABP3 and/or to corroborate the conclusion with respect to risk of PAD.

While FIG. 1 shows exemplary specific cut-offs, it will be understood that these may vary depending upon the controls selected and the size of the pool chosen as the control. For example, reaching a conclusion that a subject is highly unlikely to have PAD may be based on a cut-off of FABP3 of, for example, <0.6 ng/ml, <0.7 ng/ml, <0.8 ng/ml, <0.9 ng/ml, <1.0 ng/ml, <1.1 ng/ml, <1.2 ng/ml, <1.3 ng/ml, <1.4 ng/ml, <1.5 ng/ml, <1.6 ng/ml, <1.7 ng/ml, <1.8 ng/ml, <1.9 ng/ml, <2.0 ng/ml, <2.1 ng/ml, or <2.2 ng/ml.

Similarly, reaching a conclusion that a subject is at moderate risk of having PAD may be based on a cut-off range of FABP3 of, for example, ≥0.6 ng/ml and <4.5 ng/ml, such as ≥0.6 ng/ml, ≥0.7 ng/ml, ≥0.8 ng/ml, ≥0.9 ng/ml, ≥1.0 ng/ml, ≥1.1 ng/ml, ≥1.2 ng/ml, ≥1.3 ng/ml, ≥1.4 ng/ml, ≥1.5 ng/ml, ≥1.6 ng/ml, ≥1.7 ng/ml, ≥1.8 ng/ml, ≥1.9 ng/ml, ≥2.0 ng/ml, ≥2.1 ng/ml, or ≥2.2 ng/ml, and <3.5 ng/ml, <3.6 ng/ml, <3.7 ng/ml, <3.8 ng/ml, <3.9 ng/ml, <4.0 ng/ml, <4.1 ng/ml, <4.2 ng/ml, <4.3 ng/ml, <4.4 ng/ml, and <4.5 ng/ml, for example.

Similarly, reaching a conclusion that a subject is at moderate-high risk of having PAD may be based on a cut-off range of FABP3 of, for example, ≥3.5 ng/ml and <5.3 ng/ml, such as ≥3.5 ng/ml, ≥3.6 ng/ml, ≥3.7 ng/ml, ≥3.8 ng/ml, ≥3.9 ng/ml, ≥4.0 ng/ml, ≥4.1 ng/ml, ≥4.2 ng/ml, ≥4.3 ng/ml, ≥4.4 ng/ml, or ≥4.3 ng/ml, and <4.4 ng/ml, <4.5 ng/ml, <4.6 ng/ml, <4.7 ng/ml, <4.8 ng/ml, <4.9 ng/ml, <5.0 ng/ml, <5.1 ng/ml, <5.2 ng/ml, and <5.3 ng/ml, for example.

Similarly, reaching a conclusion that a subject is at high risk of having PAD may be based on a cut-off of FABP3 of, for example ≥4.6 ng/ml, ≥4.7 ng/ml, ≥4.8 ng/ml, ≥4.9 ng/ml, ≥5.0 ng/ml, ≥5.1 ng/ml, ≥5.2 ng/ml, or ≥5.3 ng/ml.

In aspects, reaching a conclusion that a subject is at risk of having PAD may be based on a cut-off of FABP4 of, for example, <15 ng/ml, <16 ng/ml, <17 ng/ml, <18 ng/ml, <19 ng/ml, <20 ng/ml, <21 ng/ml, <22 ng/ml, <23 ng/ml, <24 ng/ml, or <25 ng/ml.

It will be appreciated that these cut-off measurements are based on plasma FABP3 and/or FABP4 protein. It will be understood, as described below, urine FABP3 and/or FABP4 as well as RNA or DNA could be measured instead and there will be likely changes in these cut-off values, which could be calculated by a skilled person based on the teachings herein.

Typically, it is the protein biomarker that is measured. It is also possible to measure mRNA using known methods. Typically, the protein biomarkers are measured using antibodies, for example, in an ELISA or Luminex-based method. Methods for detecting and measuring the biomarkers are known to a skilled person and certain typical methods are exemplified herein.

For example, the expression pattern in blood, serum, urine etc. of the biomarkers provided herein is obtained. The quantitative data associated with the biomarkers of interest can be any data that allows generation of a useful result, including measurement of DNA or RNA levels associated with the markers but is typically protein expression patterns. Protein levels can be measured via any method known to those of skill in the art that generates a quantitative measurement either individually or via high-throughput methods as part of an expression profile. For example, a blood-derived patient sample, e.g., blood, plasma, or serum, or a urine-derived sample may be applied to a specific binding agent or panel of specific binding agents to determine the presence and quantity of the protein markers of interest.

The quantitative data associated with the biomarkers of interest typically takes the form of an expression profile. Expression profiles constitute a set of relative or absolute expression values for a number of biomarker products corresponding to the plurality of markers evaluated. In various embodiments, expression profiles containing expression patterns of at least about 2, 3, 4, 5, 6, 7, 8 or more markers are produced. The expression pattern for each differentially expressed component member of the expression profile may provide a particular specificity and sensitivity with respect to predictive value, e.g., for diagnosis, prognosis, monitoring treatment, etc.

Numerous methods for obtaining expression data are known, and any one or more of these techniques, singly or in combination, are suitable for determining expression patterns and profiles in the context of the present disclosure.

For example, DNA and RNA (mRNA, pri-miRNA, pre-miRNA, miRNA, precursor hairpin RNA, microRNP, and the like) expression patterns can be evaluated by northern analysis, PCR, RT-PCR, Taq Man analysis, FRET detection, monitoring one or more molecular beacon, hybridization to an oligonucleotide array, hybridization to a cDNA array, hybridization to a polynucleotide array, hybridization to a liquid microarray, hybridization to a microelectric array, cDNA sequencing, clone hybridization, cDNA fragment fingerprinting, serial analysis of gene expression (SAGE), subtractive hybridization, differential display and/or differential screening. These and other techniques are well known to those of skill in the art.

The present disclosure includes nucleic acid molecules, typically in isolated form. As used herein, a nucleic acid molecule is to be “isolated” when the nucleic acid molecule is substantially separated from contaminant nucleic acid molecules encoding other polypeptides. The term “nucleic acid” is defined as coding and noncoding RNA or DNA. Nucleic acids that are complementary to, that is, hybridize to, and remain stably bound to the molecules under appropriate stringency conditions are included within the scope of this disclosure. Such sequences exhibit at least 50%, 60%, 70% or 75%, typically at least about 80-90%, more typically at least about 92-94%, and even more typically at least about 95%, 98%, 99% or more nucleotide sequence identity with the sequences for the biomarkers disclosed herein, and include insertions, deletions, wobble bases, substitutions, and the like. Further contemplated are sequences sharing at least about 50%, 60%, 70% or 75%, typically at least about 80-90%, more typically at least about 92-94%, and most typically at least about 95%, 98%, 99% or more identity with the biomarker sequences disclosed herein

Specifically contemplated within the scope of the disclosure are genomic DNA, cDNA, RNA (mRNA, pri-miRNA, pre-miRNA, miRNA, hairpin precursor RNA, RNP, etc.) molecules, as well as nucleic acids based on alternative backbones or including alternative bases, whether derived from natural sources or synthesized.

The present disclosure further provides fragments of the disclosed nucleic acid molecules and/or proteins. As used herein, a fragment of a nucleic acid molecule refers to a small portion of the coding or non-coding sequence. The size of the fragment will be determined by the intended use. For example, if the fragment is chosen so as to encode an active portion of the protein, the fragment will need to be large enough to encode the functional region(s) of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming.

Protein expression patterns can be evaluated by any method known to those of skill in the art which provides a quantitative measure and is suitable for evaluation of multiple markers extracted from samples such as one or more of the following methods: ELISA sandwich assays, flow cytometry, mass spectrometric detection, calorimetric assays, binding to a protein array (e.g., antibody array), or fluorescent activated cell sorting (FACS).

In one embodiment, an approach involves the use of labeled affinity reagents (e.g., antibodies, small molecules, etc.) that recognize epitopes of one or more protein products in an ELISA, antibody-labelled fluorescent bead array, antibody array, or FACS screen. Methods for producing and evaluating antibodies are well known in the art.

A number of suitable high throughput formats exist for evaluating expression patterns and profiles of the disclosed biomarkers. Typically, the term high throughput refers to a format that performs at least about 100 assays, or at least about 500 assays, or at least about 1000 assays, or at least about 5000 assays, or at least about 10,000 assays, or more per day. When enumerating assays, either the number of samples or the number of markers assayed can be considered.

Numerous technological platforms for performing high throughput expression analysis are known. Generally, such methods involve a logical or physical array of either the subject samples, or the protein markers, or both. Common array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., xMAP® technology from Luminex (Austin, Tex.), the SECTOR® Imager with MULTI-ARRAY® and MULTI-SPOT® technologies from Meso Scale Discovery (Gaithersburg, Md.), the ORCA™ system from Beckman-Coulter, Inc. (Fullerton, Calif.) and the ZYMATE™ systems from Zymark Corporation (Hopkinton, Mass.), miRCURY LNA™ microRNA Arrays (Exiqon, Woburn, Mass.).

Alternatively, a variety of solid phase arrays can favorably be employed to determine expression patterns in the context of the disclosed methods, assays and kits. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to a, member of the candidate library, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof, that specifically interact with expression products of individual components of the candidate library are affixed to the chip in a logically ordered manner, i.e., in an array. In addition, any molecule with a specific affinity for either the sense or anti-sense sequence of the marker nucleotide sequence (depending on the design of the sample labeling), can be fixed to the array surface without loss of specific affinity for the marker and can be obtained and produced for array production, for example, proteins that specifically recognize the specific nucleic acid sequence of the marker, ribozymes, peptide nucleic acids (PNA), or other chemicals or molecules with specific affinity.

Microarray expression may be detected by scanning the microarray with a variety of laser or CCD-based scanners, and extracting features with numerous software packages, for example, IMAGENE™ (Biodiscovery), Feature Extraction Software (Agilent), SCANLYZE™ (Stanford Univ., Stanford, Calif.), GENEPIX™ (Axon Instruments).

High-throughput protein systems include commercially available systems from Ciphergen Biosystems, Inc. (Fremont, Calif.) such as PROTEIN CHIP™ arrays, and FASTQUANT™ human chemokine protein microspot array (S&S Bioscences Inc., Keene, N.H., US).

Quantitative data regarding other dataset components, such as clinical indicia, metabolic measures, and genetic assays, can be determined via methods known to those of skill in the art.

Various analytic processes for obtaining a result useful for diagnosing or staging PAD are described herein, however, one of skill in the art will readily understand that any suitable type of analytic process is within the scope of this disclosure.

### Methods

In aspects, the biomarkers described herein such as FABP3 and/or FABP4 find use in different aspects associated with diagnosing or staging PAD. FIG. 1 shows one exemplary embodiment by which FABP3 may be used clinically. In FIG. 1, an asymptomatic patient or a patient with lower limb pain presents to the clinic and FABP3 levels are measured from, for example, a blood or urine sample. Depending upon the measured amount of FABP3, the likelihood of the patient having PAD is estimated and further referrals, monitoring, or other testing is recommended. Exemplary cut-off values are shown in FIG. 1, however, it will be understood that these are not limiting and other values may be used, as explained above.

Described herein are methods for diagnosing PAD in a subject. The method typically comprises detecting the level of FABP3 in the subject; wherein an elevated level of FABP3 and/or FABP4 is indicative of PAD in the subject.

Also described herein are methods for staging PAD in a subject. Typically, the method comprises detecting the level of FABP3 and/or FABP4 in the subject; wherein an elevated level of FABP3 and/or FABP4 correlates with the stage of PAD in the subject.

Further described herein are methods for assessing arterial revascularization in a subject with PAD. Typically, the method comprises detecting the level of FABP3 and/or FABP4 in the subject; wherein a substantially normal level of FABP3 and/or FABP4 or a reduction in pre-operative elevated level of FABP3 and/or FABP4 is indicative of arterial revascularization in the subject.

Further described herein are methods for predicting whether a subject with PAD is likely to progress to CTLI. Typically, the method comprises detecting the level of FABP3 and/or FABP4 in the subject; wherein the extent of elevation of FABP3 and/or FABP4 is correlated with the likelihood of the subject progressing to CTLI.

In the methods described herein, typically the level of FABP3 and/or FABP4 is assessed as being elevated, normal, or reduced, by comparing the detected level of FABP3 and/or FABP4 to a control level of FABP3 and/or FABP4. For example, typically, the control level of FABP3 and/or FABP4 is a predetermined value obtained from one or a pool of non-PAD patients or healthy patients.

When assessing arterial revascularization, the control level of FABP3 and/or FABP4 may be the level of FABP3 and/or FABP4 that was detected in the subject prior to revascularization treatment. Thus, in aspects, the methods may comprise diagnosing and/or staging PAD by measuring FABP3 and/or FABP4 levels in the subject, initiating revascularization in subjects diagnosed with PAD, and then subsequently assessing the success and/or extent of the revascularization achieved in the subject by again measuring FABP3 and/or FABP4 levels in the subject and comparing the levels at diagnosis with the levels after treatment. If the levels have reduced over this time period, it can be concluded that revascularization is likely to have taken place to some extent. FABP3 and/or FABP4 may be measured in an ongoing manner over time to assess vascularization and/or PAD in the subject.

It will be understood that in the methods described herein, the PAD that may be diagnosed, staged, and/or treated may be non-symptomatic (stage 0), mild PAD (stage 1), moderate PAD (stage 2), severe PAD (stage 3), early CTLI (stage 4), or advanced CTLI (stages 5-6). Typically, the PAD is asymptomatic, symptomatic or advanced CTLI.

Many subjects afflicted with or suspected of being afflicted with PAD may suffer from other concurrent disorders. In aspects, the subject is free of clinical and/or biochemical evidence of myocardial ischemia, which may be determined by detecting the level of high sensitivity troponin, TnI and/or TnT in the subject. A substantially normal level of high sensitivity troponin, TnI and/or TnT in the subject suggests that the subject is free of myocardial ischemia and is further indicative of PAD in the subject. Typically, the level of high sensitivity troponin, TnI and/or TnT in the subject is determined as being substantially normal by comparing the detected level of high sensitivity troponin, TnI and/or TnT to a control level of high sensitivity troponin, TnI and/or TnT.

In additional or alternate aspects, the subject is free of clinical and/or biochemical evidence of kidney dysfunction, which may be determined by detecting the level of creatinine in the subject. A substantially normal level of creatinine in the subject suggests that the subject is free of kidney dysfunction and is further indicative of PAD in the subject. Typically, the level of creatinine in the subject is determined as being substantially normal by comparing the detected level of creatinine to a control level of creatinine.

Likewise, the subject being assessed using the methods described herein may be free of clinical and/or biochemical evidence of acute stroke and/or acute muscle toxicity.

On the other hand, as noted above, the subject may have a concurrent condition that would typically be expected to confuse the diagnosis of PAD. However, it has been found herein that FABP3 and/or FABP4 is still a good predictor of PAD despite the presence of these conditions, even without adjusting the detected level of FABP3 and/or FABP4 and/or the control level of FABP3 and/or FABP4. In these cases where concurrent conditions may exist in the subject, the detected level of FABP3 and/or FABP4 and/or the control level of FABP3 and/or FABP4 is optionally adjusted for the concurrent condition. Typically, the concurrent condition is kidney dysfunction, stroke, diabetes, and/or muscle toxicity.

It will be understood that the biomarkers described herein may be detected in any bodily fluid or tissue in which it is expressed. For example, the biomarkers may be detected in whole blood, plasma, urine, muscle tissue, saliva, oral fluid, cerebrospinal fluid, amniotic fluid, milk, colostrum, mammary gland secretion, lymph, sweat, lacrimal fluid, gastric fluid, synovial fluid, mucus, or combinations thereof. Typically, the biomarkers are detected in blood, urine, or a biopsy sample.

Likewise, as described above, the biomarkers described herein may be detected in any form, including protein, DNA, RNA, or a combination thereof. Typically, the biomarkers are detected as protein.

It will be understood that the biomarkers may be assessed in any known age group suspected of being afflicted with PAD. Typically, the subject is an adult and is typically at least 20, 25, 30, 35, 40, 45, 50, 55, 60, 65, 70, 75, or 80 years of age.

The methods described herein also encompass treating the subject based upon the outcome of the method. For example, if the subject is diagnosed with early CTLI, the subject may be further screened and referred for treatment. Thus, also described herein are methods of treating a subject with peripheral artery disease. The methods of treatment comprise carrying out a diagnostic or staging method described herein and treating the subject based upon the diagnosis or stage of disease.

The above disclosure generally describes the present invention. A more complete understanding can be obtained by reference to the following specific examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention and are not to be construed as limiting in any way the remainder of the disclosure.

### EXAMPLES

### Example 1: Fatty Acid-Binding Protein 3 (FABP3): A Novel Biomarker for the Diagnosis of Peripheral Arterial Disease

### Abstract

Of the 200 million people that suffer from peripheral artery disease (PAD) worldwide, 5-25% progress to chronically threatened limb ischemia (CTLI), which has devastatingly high rates of lower limb amputations and mortality. Unfortunately, the diagnosis of CTLI is often delayed, which results in an increased risk of limb loss, morbidity, and mortality. The purpose of this study was to identify circulating blood biomarker(s) for use alongside clinical assessments to diagnose PAD including patients with CTLI.

In this study, ELISA experiments were conducted on non-PAD (n=40) and CTLI patients (n=50) to investigate the levels of Fatty Acid Binding protein 3 (FABP3), also referred to as heart type FABP (hFABP). Binary logistic regression analysis was also conducted while controlling for confounding factors. Receiver operating characteristic (ROC) curves, alongside the non-parametric estimate of the area under the curve (AUC), and their corresponding 95% CI were calculated. Our data demonstrated that FABP3 is significantly up-regulated in CTLI patients when compared to the non-PAD control group, even after adjusting for confounding risk factors. FABP3 has a large effect size for CTLI when compared to non-PAD patients, an associated OR of 1.8, an AUC of 0.8, as well as a significant reduction in its plasma levels after arterial revascularization. Lastly, FABP3 levels were noted to increase with worsened PAD severity with the absence of clinical and biochemical evidence of myocardial ischemia.

Our findings demonstrate that FABP3 is a potential biomarker that can be used to diagnose patients with PAD as well as CTLI. This will significantly curtail the morbidity and mortality associated with this disease.

### Introduction

Lower extremity peripheral arterial disease (PAD) is a common presentation of atherosclerosis¹. Over 200 million people are reported to have the disease², and the global prevalence of PAD is estimated to range from 3-12%^{1 3}. However, despite its prevalence, PAD goes undiagnosed commonly, with studies suggesting that physicians fail to detect and diagnose PAD in 51% of their patients⁴. This lack of PAD awareness among healthcare providers and patients results in the failure of medical therapy initiation, atherosclerosis risk factor modification, and deferred referral to specialists. Moreover, it also leads to a delay in surgical intervention, which puts patients at significant risk of morbidity, disability, mortality and major limb amputation.

Furthermore, it is estimated that over a period of five years, 5-25% of PAD patients experience disease progression to the most severe form of PAD, known as critical limb ischemia (CTLI)⁵⁻⁸. CTLI manifests as rest-pain (early stage CTLI), or non-healing ischemic ulcers with gangrenous tissue loss in the lower extremities (late stage CTLI)⁹. Currently, treatment options for CTLI are limited to medical management in addition to arterial revascularization or major limb amputation¹⁰. However, despite our best efforts, patients with CTLI still face a high mortality and disability risk. The Trans-Atlantic Inter-Society Consensus for the Management of Peripheral Arterial Disease (TASC II) report estimates a 25% mortality rate at one year for patients diagnosed with CTLI⁹. Similarly, a review of 20,464 patients who underwent a major amputation secondary to CTLI demonstrated that a significant portion of patients had no surgical attempts performed on them in the year prior to amputation¹¹.

One major reason for these high rates of lower limb amputation and deaths is delayed diagnosis. This is exemplified in a recent study which showed that primary amputation was the first treatment performed for 67% of Medicare patients with CTLI²². However, several studies have suggested that a significant portion of lower limb amputations can been delayed or prevented, especially through early diagnosis and routine screening¹³⁻¹⁵.

However, identifying patients in the early stages of CTLI is a major challenge faced by many physicians and primary healthcare providers. The ratio of the brachial artery blood pressure to the ankle blood pressure—ankle brachial index (ABI)—serves as a cost-effective screening tool for PAD^{16, 17}. However, it fails to reliably diagnose PAD in many patients as well as patients with CTLI^{16, 17}. Moreover, ABI values of diabetic patients, with and without PAD, are usually falsely elevated due to incompressible calcified vessels¹⁸. Thus, the ABI ratio is not a reliable tool in being able to distinguish patients with PAD from within a large overall proportion of patients with suspected lower limb pain¹⁹. Furthermore, while a few risk-prediction models for PAD have been developed²⁰⁻²³, these models currently lack rigorous external data validation and at best have demonstrated modest predictive abilities. As a result, they are not widely used in clinical practice²⁴. Circulating biomarkers such as β-2-microglobulin and C-Reactive Protein (CRP) have also been proposed to be indicative of PAD status^{25 26}, however, they lack the appropriate specificity and sensitivity required to diagnose PAD or CTLI²⁷.

Consequently, distinguishing patients with PAD still remains a major diagnostic challenge for clinicians today. This is especially true for physicians with limited access to medical specialists and diagnostic imaging. Therefore, a purpose of this study was to characterize the proteomic plasma profile of patients with PAD as well as CTLI in order to identify a biomarker(s) that can assist physicians in diagnosing PAD and CTLI.⁹⁻¹³.

### Material and Methods

### Study Design, Setting, and Data Sources

We conducted a case-control study at St. Michael's Hospital (Toronto, Canada) with PAD and CTLI patients serving as cases and non-PAD patients serving as controls.

### Ethics Approval

This study was approved by the research ethics board at St. Michael's Hospital—University of Toronto in Ontario, Canada. Informed consent was obtained from all participants. All the experiments performed were conducted in accordance to the relevant guidelines and regulations.

### Patient Selection

The PAD status was defined clinically as per the Rutherford Classification Criteria of chronic limb ischemia²⁶. Patients with PAD referred to vascular surgery ambulatory clinics or emergency department at St. Michael's Hospital from June 2017 through March 2018 were asked to participate in this study. We excluded all patients on anticoagulants, chemotherapy or biological anti-inflammatory agents. Patients diagnosed with sepsis, systematic inflammatory disease or with active/history of any cancer or deep vein thrombosis (DVT) were excluded as well. Moreover, patients with an acute or 6 month history of acute coronary syndrome, heart failure, or uncontrolled arrhythmia as defined by American College of Cardiology, also failed to meet the inclusion criteria of this study¹⁸⁻²³. The non-PAD control cohort was defined as patients with cardiovascular risk factors alongside a normal arterial US of the lower limbs, palpable distal pulses and without a significant clinical history of claudication.

### Baseline Measurements

In all subjects, a thorough physical exam and complete medical history was obtained from each patient by an independent PAD expert. Medical history including details of any previous acute coronary syndrome, hyperlipidemia, arterial arrhythmia, arterial hypertension, renal disease, congestive heart failure, history of stroke or transient ischemic attack (TIA), history of cancer, diabetes, and smoking status. Each patient received lower limb arterial imaging (arterial ultrasound (US), computed tomography angiogram (CTA) or angiogram) as part of the PAD assessment. Arterial US findings including ankle brachial index (ABI) were recorded for each patient. Blood samples were drawn into vacutainer tubes containing EDTA. Plasma was then extracted from this blood via centrifugation at 3000 rpm for 10 min (4° C.), which was then aliquoted and stored at −80° C. Plasma samples that had previously been thawed were not utilized for this study.

### Study Size and Bias

In this study, plasma samples were collected from 40 non-PAD controls and 50 CTLI patients (Table 1). This sample size allowed us to detect a difference between non-PAD controls and CTLI patients of 0.50 standard deviation units in FABP3 with 80% power at a two-sided alpha of 0.05. Since our analyses were exploratory, alpha levels were not adjusted for multiple comparisons.

TABLE 1
Baseline Characteristics for the patient Cohort by subgroup
(N = 90).

| | Baseline Characteristics | | |
|---|---|---|---|
| | Non-PAD | CTLI | p-value |
| Number of patients | 40 | 50 | |
| Age, (yrs) | 62 | 70 | <0.001* |
| Male (%) | 70 | 69 | 0.563 |
| Hypertension (%) | 72 | 88 | 0.014* |
| Hypercholesterolemia (%) | 62 | 85 | 0.003* |
| Diabetes mellitus (%) | 46 | 57 | 0.106 |
| Renal Insufficiency (%) | 2 | 12 | 0.061 |
| Smoking (%) | 58 | 85 | 0.010* |
| Congestive heart failure (%) | 0 | 7 | 0.086 |
| Coronary artery disease (%) | 16 | 52 | <0.001* |
| Stroke/TIA (%) | 6 | 16 | 0.122 |
| Ankle pressure (mmHg) | 109 | 38 | <0.001* |

Significant values are noted with an (*) for P value ≤ 0.05 Outcomes

A primary outcome of this study was to confirm FABP3 a potential biomarker for PAD and CTLI that could be used for diagnostic purposes. Secondary outcomes included identifying the source of this biomarker, as well as its effects after arterial revascularization, among others.

### Patient Selection for Proteomics Protein Discovery

The non-PAD control cohort were defined as patients with cardiovascular risk factors including hypertension, hyperlipidemia, diabetes, smoking, or family history of heart disease, alongside a normal arterial US of the lower limbs and palpable distal pulses, but without a significant clinical history of PAD claudication. The PAD patients were defined clinically as per the Rutherford Classification Criteria of chronic limb ischemia²⁸. Asymptomatic patients had no clinical symptoms of PAD however these patients had radio-graphical evidence of PAD on ultrasound (stage 0). Mild PAD patients had mild claudication and were able to complete a treadmill exercise test (five minutes walking on a treadmill at 2 mph on a 12% incline) with ankle pressure (AP) of >50 mm Hg after exercise. However, the AP needed to be at least 20 mmHg lower than resting value (stage 1). The moderate PAD patients had moderate claudication and were between stages 1-3 (stage 2). The severe PAD patients had disabling claudication and were not able to complete the treadmill exercise with an AP after exercise of <50 mmHg (stage 3). In contrast, the early CTLI patients (stage 4) were defined clinically as patients with the presence of ischemic rest pain and the absence of ischemic ulcers or gangrenous tissue. These patients had a resting AP of <40 mmHg with a toe pressure (TP) of <30 mmHg, as well as an evidence of severe arterial disease documented on angiogram or CTA.

The advanced CTLI patients had evidence of tissue loss as well as AP of <40 mmHg with a TP of <30 mmHg (stages 5-6). Lastly, we recruited a group of healthy patients, which served as the negative control in some experiments. This group was defined as participants without any cardiovascular risk factors, alongside a normal arterial US of the lower limbs.

The following patients were excluded:

1) Patients on chemotherapy or biological anti-inflammatory agents.

2) Patients diagnosed with sepsis, inflammatory disease or with active/history of any cancer.

3) Patients with an acute or 6 month history of acute coronary syndrome, heart failure, or uncontrolled arrhythmia as defined by American College of Cardiology²⁹⁻³².

4) Patients diagnosed with acute limb ischemia.

5) Patients with tissue loss/gangrene (late CTLI Rutherford stages 5-6) were excluded from this study, as these patients have advanced disease and tissue ischemia.

### FABP3 and Troponin Multiplex Assay

This experiment was carried out in order to simultaneously assess FABP3 and Troponin I (TnI) levels in 90 non-PAD and CTLI patients. Patients' arterial status was classified as described above. We also recruited patients that presented to our cardiac care unit with documented acute coronary disease (ACS) on ECG and angiogram to serve as a positive control cohort.

Plasma samples were analyzed in duplicate using MILLIPLEX MAP Human Cardiovascular Disease (CVD) Magnetic Bead Panel 1 (EMD-Millipore; Billerica, Mass.) to determine the concentrations of FABP3 and TnI. Analysis was completed as described by the manufacturer. All sample analyses were completed on the same day to eliminate inter-assay variability. Sample intra-assay and inter-assay CV were both <10%. The MagPix analyzer (Luminex Corp; Austin, Tex.) was calibrated prior to analysis using Fluidics Verification and Calibration bead kits (Luminex Corp). A minimum of 50 beads for each targeted biomarker were acquired using Luminex xPonent software and analyzed using Milliplex Analyst software (v.5.1; EMD-Millipore).

Furthermore, in order to study the relationship between FABP3 and PAD, FABP3 levels were measured in a 486 non-PAD and PAD patients. Lastly, in order to study the relationship between FABP3 and PAD severity status, FABP3 levels were measured in a group of 250 patients stratified by their PAD status (PAD and CTLI) along with non-PAD patients who served as a negative control. Once again, samples were measured in duplicate using MILLIPLEX MAP CVD Magnetic Bead Panel 1 kit, as described above

### Plasma Levels of Biomarkers in CTLI Before and After Arterial Revascularization

In the CTLI cohort, twelve patients who had undergone infra-inguinal bypass arterial revascularization agreed to provide blood samples pre-operatively and post-operatively. The post-operative sample was collected twelve weeks after surgery. Blood samples were processed as aforementioned and used to assess the biomarker before and after surgery via an protein multiplex.

### Muscle Homogenate Preparation and Western Blot Analysis

To localize our biomarker within skeletal muscles, gastrocnemius muscle was obtained from patients with end stage CTLI undergoing major limb amputation (experimental group n=4) and healthy non-PAD patients undergoing elective orthopedic surgeries (negative control group n=3). Immunoblots were carried out using homogenate skeletal muscle protein obtained both groups. All snap frozen muscle samples were homogenized in a lysis buffer (Cell Signaling Technology, Beverly, Mass., USA) using a ultrasonic homogenizer (Biologics Inc, Manassa, Va., USA). Protein concentration was measured in duplicate by the bicinchoninic acid (BCA) assay (Pierce, Rockford, Ill. USA). For the Western blot studies, aliquots equivalent of approximately 80 μg of protein were separated on gradient (10-15%) polyacrylamide sodium dodecyl sulphate gels. After electrophoresis, proteins were electro-transferred to a nitrocellulose membrane. The membrane was blocked with skimmed milk powder in TBST (0.05% Tween 20, 100 mM NaCl, 10 mM Tris-HCl pH 7.8) for 30 minutes and then incubated with the primary antibody FABP3 (Abcam, Toronto, ON, Canada) overnight at 4° C. Secondary antibody polyclonal goat anti-mouse horseradish peroxidase (HRP) conjugated (Cedarlane, Burlington, ON, Canada) was added. Blots were developed in ECL detection reagents (Amersham, ECL Western Blotting Detection Reagents, GE Healthcare) and the chemiluminescence emitted from immune complexes was visualized with a ChemiDoc image system (Bio-Rad, Mississauga, ON, Canada). The images were quantified by Image J software.

### Immunohistochemistry of FABP3 Biomarkers

Immunohistochemistry was performed on 5 μm formalin-fixed, paraffin-embedded muscles obtained from lower limb amputations performed to treat CTLI (n=4). Non-ischemic gastrocnemius muscle was obtained from non-PAD patients undergoing elective orthopedic surgeries not related to CTLI (n=3). Hematoxylin-eosin staining and Masson's Trichrome were conducted according to manufacturer's instructions (Sigma, St. Louis, Mo., USA). For detection of FABP3 and CD68, sections were stained using anti-human mouse polyclonal antibody (Thermo Fisher Scientific, Massachusetts). These sections were incubated at 4° C., and then incubated again with HRP-conjugated secondary antibodies according to the immunostaining procedure.

### Statistical Methods

Demographics and baseline measurements were recorded for each patient. Baseline data were expressed as means with standard deviations (SD) or as percentages. Evaluations of baseline characteristics were done using independent t-tests or Mann-Whitney U test for continuous variables. Fisher's exact test or chi-square test was used for categorical variables. ANOVA was used in experiments where more than two group differences needed to be analyzed. Cohen's d was used to compute the effect size for the comparison between two group means. Treatment outcomes across the groups or according to specific biomarkers were analyzed with logistic regression analyses. A stepwise binary logistic regression analysis using the backward elimination procedure was performed to study the impact of potential confounders. Confounding variables were identified to be age, gender, smoking, diabetes mellitus, coronary artery disease, hypertension, hypercholesterolemia and statins, as per our literature review^{1-3, 5, 33, 34}. For significantly associated biomarkers, receiver operator characteristic (ROC) curves were estimated, which served as a visual means to describe the ability of the model to correctly classify ‘CTLI or PAD’ and ‘non-PAD’ patients. Non-parametric estimate of the area under the curve (AUC), Youden index and their corresponding 95% CI were calculated SPSS software version 23 (SPSS Inc., Chicago, Ill., USA) was used for data entry and analysis, while Prism 7 (Graphpad, San Diego, Calif., USA) was used for various graphical illustrations and volcano plots. All analyses were carried out at a 5% two-sided significance level.

### Results

### ELISA Confirmation of Candidate Biomarkers

To confirm the up-regulation of FABP3 in patients with PAD, protein levels were measured in CTLI and non-PAD patients (Table 1). We identified significant differences in the demographics of both patient groups with regards to age, hypertension, hypercholesterolemia, history of smoking, coronary arterial disease and ABI values. After conducting in-depth statistical analysis, FABP3 had a large effect size with a corresponding value of 1.02 as well as a large mean difference of 2.51 ng/ml (95% CI 1.17-3.86) (Table 2). In order to study the association between FABP3 and CTLI, we calculated the odds ratio (OR). Furthermore, to account for the effect of confounding factors, we conducted binary logistic regression analysis accounting for age, gender, smoking, diabetes mellitus, hypertension, hypercholesterolemia, coronary arterial disease, chronic kidney disease and statin usage (Table 3). FABP3 was found to have a large OR (1.88, 95% CI 1.45-2.37), which remained significant even after adjusting for confounding factors (1.42, 95% CI 1.04-1.93).

TABLE 2
Protein multiplex studies confirming the up-regulated of
FABP3 in CTLI patients.

| | | Mean Difference | |
|---|---|---|---|
| Biomarker | Effect Size | (ng/ml) | 95% CI |
| FABP3 | 1.02 | 2.51* | 1.17-3.86 |

Significant values are noted with an (*) for P value ≤ 0.05

TABLE 3
Unadjusted and adjusted odds ratio after conducting logistic regression
analysis. Binary Logistic regression reference category is non-PAD.
Adjusted regression analysis were conducted for the following
confounding variables: age, gender, smoking, diabetes mellitus,
hypertension, hypercholesterolemia, coronary artery disease, chronic
kidney disease and statins.

| | Unadjusted Odds Ratios | | Adjusted Odds Ratio | |
|---|---|---|---|---|
| | OR | 95% CI | OR | 95% CI |
| FABP3 | 1.875 | 1.454-2.373 | 1.418 | 1.042-1.929 |

### Plasma Levels of FABP3 in CTLI Before and After Arterial Revascularization

For deeper insights on the biochemical effects of arterial revascularization on the FABP3, the levels of each protein were measured in twelve CTLI patients prior to surgery and three months following surgery via protein multiplex (Table 4). Our data shows that arterial revascularization of the ischemic limb causes a significant down regulation in circulatory levels of FABP3.

TABLE 4
Biomarker level in the same CTLI patients before and after arterial
revascularization. Plasma levels of FABP3 were measured in twelve
CTLI patients before and twelve weeks after re-establishing blood flow.

| | | Mean difference | |
|---|---|---|---|
| | Effect Size | (ng/ml) | 95% CI |
| FABP3* | 0.96 | 1.2* | 0.26-2.20 |

*P value ≤ 0.05.

### Receiver Operating Characteristic (ROC) Analysis

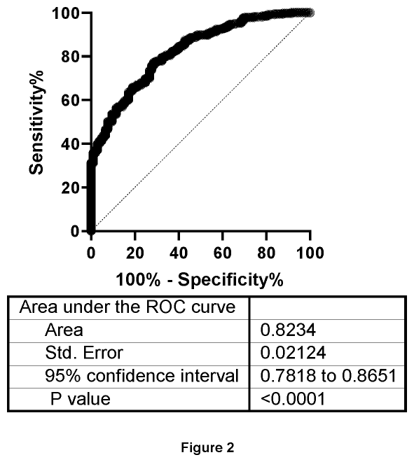

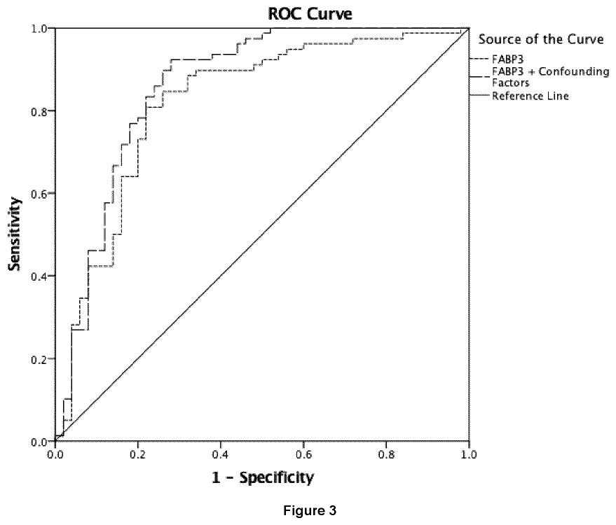

FABP3 was selected for further statistical analysis as a potential biomarker for PAD and CTLI as this protein had a large OR, large effect size, as well as level normalization after arterial revascularization. Two predictive models were compared in this ROC analysis. First, a ROC curve was estimated for FABP3 as a single predictor for PAD in a group of 486 patients, which demonstrated an area under curve (AUC) of 0.8234 (95% CI, 0.7818 to 0.8651) is represented by the solid line (FIG. 2). When compared to non-PAD control, our ROC analysis confirms the use of FABP3 in plasma as an excellent biomarker of PAD with large area under curve. Second, a ROC curve was estimated for FABP3 as a single predictor of CTLI, which demonstrated an AUC of 0.80 (95% CI 0.65-0.87) (FIG. 3). However, to further understand the prediction capabilities of FABP3, another ROC curve was estimated using probability estimates from a fitted model featuring CTLI status regressed on FABP3 and previously validated confounding factors (age, gender, smoking, diabetes mellitus, hypertension, hypercholesterolemia, coronary artery disease, chronic kidney disease and statins). The AUC value for FABP3 was improved after adjusting for confounding risk factors (0.92, 95% CI 0.79-0.97) (FIG. 3).

### FABP3 Levels in Progressive PAD Disease

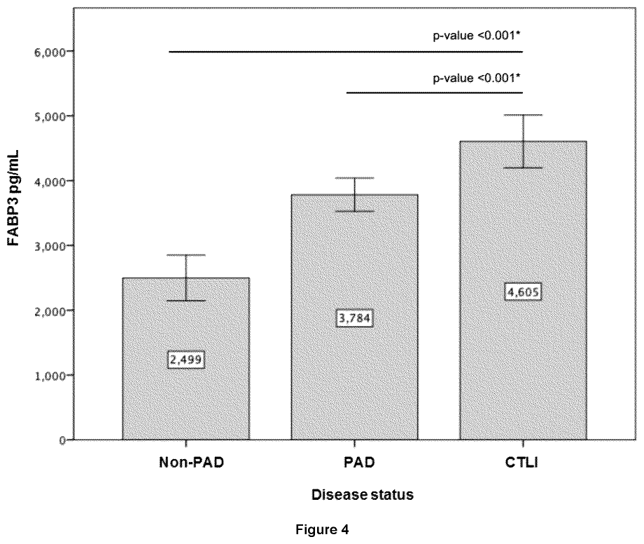

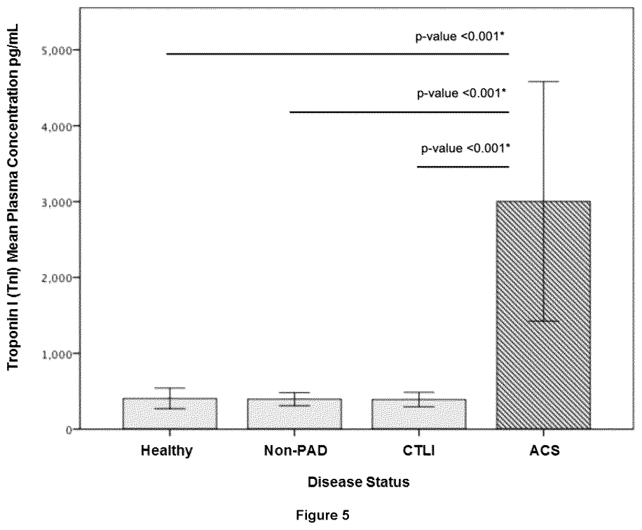

The relationship between worsening PAD severity and FABP3 was also investigated, as our ROC analysis demonstrated a significant true positive rate (TPR) for FABP3, indicating its potential as a diagnostic biomarker for PAD and CTLI. In this experiment, FABP3 levels were measured in non-PAD, PAD and CTLI patients (n=250). An increase in FABP3 levels was observed as the severity of PAD increased (FIG. 4). FABP3 also had a significantly high mean value of 4.6 ng/mL, SE 0.2 ng/mL) in CTLI patients when compared to other clinical stages (p-value<0.0001), as per our ANOVA analysis.

FABP3 Levels Increase Due to CTLI without Evidence of Myocardial Injury or Renal Failure

Although patients with ACS were excluded from this study, we wanted to assess for biochemical evidence of cardiac injury, as FABP3 is linked to cardiovascular disease. Hence, the presence of myocardial ischemia was investigated on a molecular level via protein multiplex, by simultaneously measuring the levels of troponin I and FABP3 in our control and experimental groups. Patients presenting to our hospital with ACS served as the positive control (n=15), whereas fifteen healthy patients without any cardiovascular risk factors or PAD represented the negative control group. After analyzing our data, there was no significant difference found in levels of troponin I in the CTLI cohort in comparison to the control group (FIG. 5). However, as expected, the positive ACS control group had elevated levels of TnI relative to healthy, non-PAD and CTLI patients. Given the absence of clinical signs of myocardial ischemia, in addition to similar troponin levels in the CTLI group and the negative control group, the increase in FABP3 levels observed in CTLI patients is likely not associated with myocardial injury. This experiment favours the hypothesis that the increased FABP3 plasma levels observed in PAD and CTLI patients is not a result of myocardial injury. Lastly, we did not observe any statistical difference in creatinine levels between non-PAD, PAD and CTLI cohorts (p-value=0.153), indicating that the increase in FABP3 levels in the PAD and CTLI cohort is independent of kidney dysfunction.

Localization of FABP3 within Lower Limb Skeletal Muscles

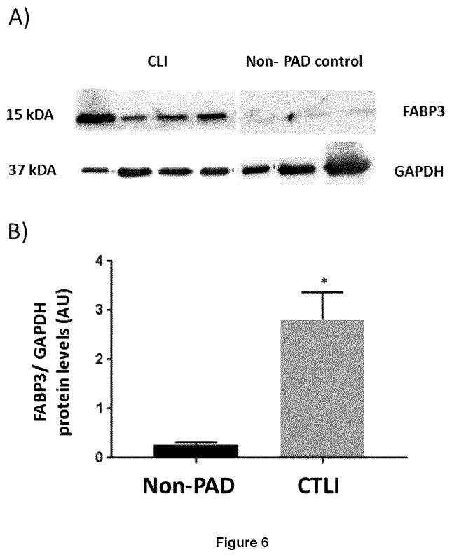

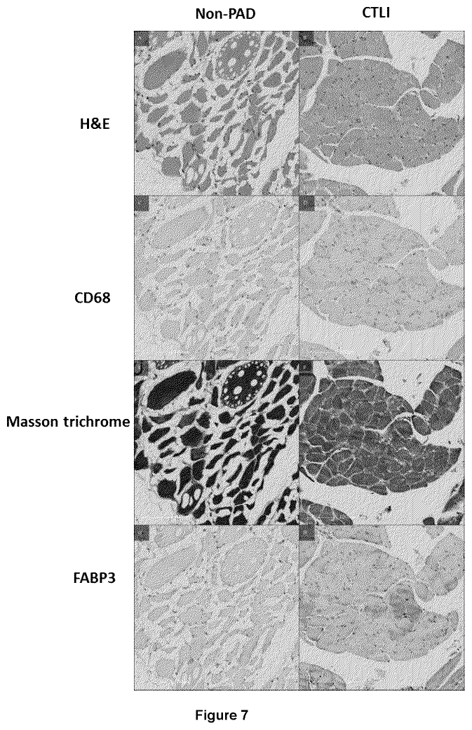

In this part of the study, gastrocnemius skeletal muscles were investigated as a possible source of FABP3 in patients with CTLI. Using Western blot studies, FABP3 expression was investigated in samples obtained from control group (non-PAD patients undergoing elective orthopedic surgeries) and experimental group (CTLI patients undergoing lower limb amputation). Quantitative analysis revealed over two-fold increase in expression of FABP3 in muscle tissue obtained from CTLI patients, when compared to controls (FIG. 6). Next, immunohistochemistry was performed on experimental and control skeletal muscles to further confirm the immune-blot findings. Higher levels of FABP3 as well as CD68 (marker of macrophages) were observed within ischemic muscles in relation to control samples. Furthermore, the Masson's trichrome stain demonstrates increased levels of tissue fibrosis within muscles isolated from CTLI patients relative to the control group. Along with the western blot findings, this data favors the hypothesis that skeletal muscles might be a potential source of FABP3 in patients with CTLI (FIG. 7).

### Discussion

In this study, we sought to confirm FABP3 as a diagnostic marker for PAD and CTLI. To achieve this, non-PAD patients, PAD and CTLI patients were investigated for the levels of FABP3 in plasma. Our data demonstrated that, relative to non-PAD controls, patients with CTLI had a large FABP3 effect size and high FABP3 OR. Moreover, FABP3 levels were observed to increase with severity of PAD, but also decrease after successful arterial revascularization. Thus, given these novel findings, FABP3 appears to be a robust biomarker for identifying patients with PAD including patients with CTLI.

FABP3, also known as heart-type FABP, belongs to a family of multigene fatty acid-binding proteins. It is primarily expressed in the heart, where it constitutes 4-5% of all cellular proteins, but is also expressed in the brain and skeletal muscle among other organs and tissues³⁵. Within muscle cells, FABP3 is primarily responsible for mediating the uptake of intracellular fatty acids as well as their transport toward the mitochondrial β-oxidation system³⁶. Moreover, elevated levels of FABP3 have been reported in patients with diabetes, muscle toxicity, among other conditions^{35, 37-39}. Recent studies have also alluded to the capabilities of FABP3 in serving as a serum biomarker for the early diagnosis of stroke and acute myocardial ischemia⁴⁰⁻⁴². Similarly, the findings from this study suggest that FABP3 also serves as a biomarker for the diagnosis of PAD as well as CTLI, after adjusting for confounding factors, including those conditions that lead to elevated levels of FABP3 (such as diabetes). This has immense clinical utility, especially in ambiguous patient cases where diagnosis of PAD is suspected, but uncertain. For instance, studies have shown that diagnosing diabetic patients with PAD or CTLI can be challenging due to neuropathy, which masks the ischemic rest pain associated with CTLI⁴³. Consequently, this makes it harder for physicians to decide if a high-risk intervention is needed or not, as these patients are only suspected to have CTLI⁴⁴. Thus, having a clinical biomarker for PAD and CTLI eliminates the ambiguity surrounding patient cases, as it can concretely indicate to physicians if an intervention is needed or not. Moreover, many CTLI patients report experiences of prolonged wait times⁴⁵. Subsequently, another clinical advantage that comes with having a biomarker for CTLI is that it significantly reduces the wait time, as it can diagnose CTLI within minutes and subsequently lead to earlier intervention.

Other studies have also explored the use of circulating protein biomarkers for CTLI, but reached vastly different conclusions in comparison to the findings from this study. For instance, Li et al., identified Siglec 5 as a biomarker of CTLI⁴⁶. However, our data does not demonstrate the overexpression of Siglec 5 in CTLI patients when compared to our control non-PAD patients. One reason for this disparity in findings could be due to differences in patient cohort selections. Li et al., only recruited diabetic patients with ABI>0.9, whereas we recruited both diabetic and non-diabetic CTLI patients with an ankle pressure of <40 mm Hg and toe pressure of <30 mm Hg²⁸. We used Rutherford's criteria of ankle and toe pressures to classify PAD patients over ABI, as the ABI values of diabetic patients with and without CTLI are usually falsely elevated due to incompressible calcified vessels¹⁸. Consequently, we did not deem the ABI ratio to be a reliable tool in being able to diagnose diabetic patients with CTLI¹⁹. Furthermore, Li et al., used a targeted “246 protein based chip assay” to identify their biomarker⁴⁶. In our study, not only did we recruit both diabetic and non-diabetic patients, but we were also the first to identify FABP3 as a candidate protein biomarker for PAD and CTLI patients. Similarly, another study by Hung and authors found over 50 differentially expressed plasma proteins for CTLI. However, they were also limited in their patient cohort as they only recruited hemodialytic diabetic patients with and without CTLI⁴⁷.

With regards to its source of expression, studies have reported that FABP3 is mainly expressed in myocardial and skeletal muscles⁴⁸⁻⁵⁰. Upon conducting western blots and immunohistochemistry, our data was consistent with past research findings and demonstrated that ischemic skeletal muscles are the likely source of FABP3 expression in CTLI patients, suggesting a relationship between the two.

### REFERENCES

1. Conte S M and Vale P R. Peripheral Arterial Disease. Heart Lung Circ. 2018; 27:427-432.
2. Fowkes F G, et al. Lancet. 2013; 382:1329-40.
3. Cornejo Del Rio V, et al. PLoS One. 2017; 12:e0186220.
4. Hirsch A T, et al. Jama. 2001; 286:1317-1324.
5. Brevetti G, et al. Angiology. 1998; 49:843-8.
6. O'Riordain D S and O'Donnell J A. Br J Surg. 1991; 78:861-3.
7. Jelnes R, et al. Br Med J (Clin Res Ed). 1986; 293:1137-40.
8. Rosenbloom M S, et al. Arch Surg. 1988; 123:867-70.
9. Norgren L, et al. Eur J Vasc Endovasc Surg. 2007; 33 Suppl 1:S1-75.
10. Dormandy J, et al. Semin Vasc Surg. 1999; 12:142-7.
11. Goodney P P, et al. Circulation: Cardiovascular Quality and Outcomes. 2012; 5:94-102.
12. Allie D E, et al. EuroIntervention: journal of EuroPCR in collaboration with the Working Group on Interventional Cardiology of the European Society of Cardiology. 2005; 1:75-84.
13. Jensen P S, et al. BMJ open. 2017; 7:e016030.
14. Vaidya A, et al. BMC Public Health. 2014; 14:89.
15. Buckley C M, et al. BMJ Open Diabetes Research and Care. 2015; 3:e000069.
16. Alahdab F, et al. J Vasc Surg. 2015; 61:42S-53S.
17. Crawford F, et al. Cochrane Database Syst Rev. 2016; 9:CD010680.
18. Peripheral arterial disease in people with diabetes. Diabetes Care. 2003; 26:3333-41.
19. Castronuovo J J, Jr., et al. J Vasc Surg. 1997; 26:629-37.
20. Meltzer A J, et al. J Vasc Surg. 2013; 57:1186-95.
21. Schanzer A, et al. J Vasc Surg. 2008; 48:1464-71.
22. Bradbury A W, et al. J Vasc Surg. 2010; 51:52S-68S.
23. Mills J L, Sr., et al. J Vasc Surg. 2014; 59:220-34 e1-2.
24. Chung J, et al. J Vasc Surg. 2014; 60:1677-85.
25. Wilson A M, et al. Circulation. 2007; 116:1396-403.
26. Owens C D, et al. J Vasc Surg. 2007; 45:2-9; discussion 9.
27. Real de Asua D, et al. Int Cardiovasc Res J. 2012; 6:107-12.
28. Rutherford R B, et al. J Vasc Surg. 1997; 26:517-38.
29. Thygesen K, et al. J Am Coll Cardiol. 2018.
30. A I-Khatib S M, et al. J Am Coll Cardiol. 2017.
31. Amsterdam E A, et al. Circulation. 2014; 130:e344-426.
32. O'Gara P T, et al. 2013 ACCF/AHA guideline for the management of S T-elevation myocardial infarction: a report of the American College of Cardiology Foundation/American Heart Association Task Force on Practice Guidelines. Circulation. 2013; 127:e362-425.
33. Bailey M A, et al. Semin Intervent Radiol. 2014; 31:292-9.
34. Hirsch A T, et al. Circulation. 2006; 113:e463-654.
35. Varrone F, et al. Journal of the American College of Cardiology. 2013; 61:88-95.
36. Furuhashi M and Hotamisligil G S. Nature reviews Drug discovery. 2008; 7:489.
37. Karbek B, et al. Cardiovascular diabetology. 2011; 10:37.
38. Beysel S, et al. 2017; 12:2063.
39. Kochansky C J, et al. Lipids. 2018; 53:947-960.
40. Vupputuri A, et al. Indian Heart J. 2015; 67:538-42.
41. Okamoto F, et al. Clin Chem Lab Med. 2000; 38:231-8.
42. Zimmermann-Ivol C G, et al. Molecular & Cellular Proteomics. 2004; 3:66-72.
43. Barraclough K and Bradbury A. BMJ. 2018; 360:j5460.
44. de Graaff J C, et al. Journal of vascular surgery. 2003; 38:528-534.
45. Duval S, et al. American heart journal. 2014; 168:577-587.
46. Li J Y, et al. Sci Rep. 2017; 7:11272.
47. Hung P H, et al. Mol Biosyst. 2011; 7:1990-8.
48. Claffey K P, et al. Biochemistry. 1987; 26:7900-4.
49. Adhikari S, et al. Mol Cell Biochem. 2007; 296:59-67.
50. Glatz J F, et al. Acta Physiol Scand. 2003; 178:367-71.

### Example 2: Detection of FABP3 in Urine and Plasma

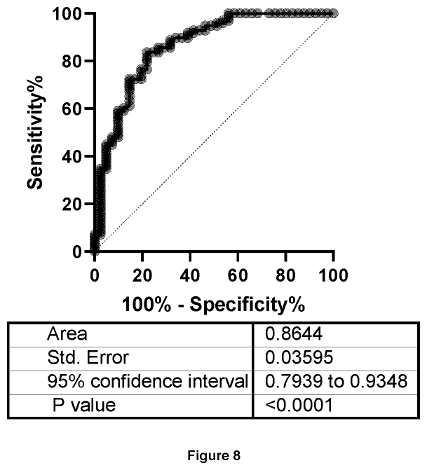

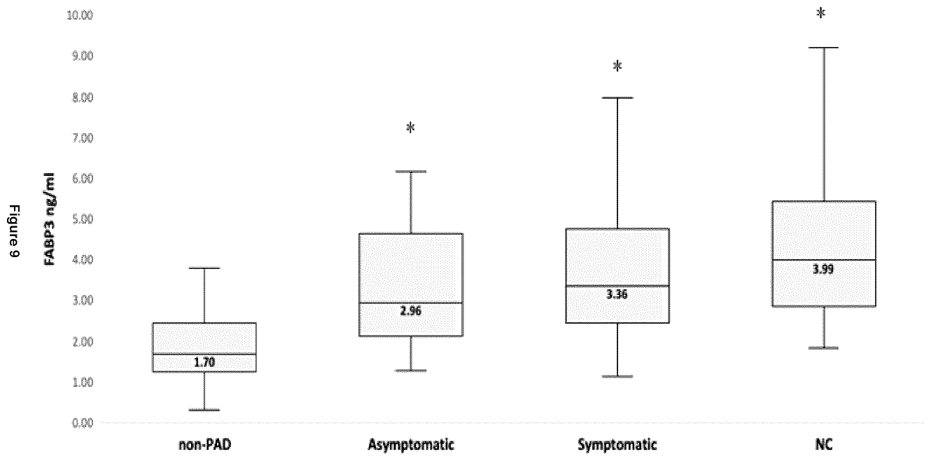

As shown in FIG. 8, we conducted ROC analysis for FABP3 in urine in non-PAD and PAD patients. When compared to non-PAD control n=41, our data confirms the presence of FABP3 in urine. Additionally, the ROC analysis demonstrates the excellent potential use of FABP3 in urine as a robust biomarker of PAD with large area under curve.

### Example 3: A Comparison of Multiple Biomarkers in Non-PAD Subjects, PAD Subjects, and ACS Subjects

We assessed the levels of TnI, FABP4, and FABP3 and assessed ABI in subjects that do not have PAD (n=114), those that do have PAD (n=391), and those that have ACS (n=15). As shown in Table 5, FABP4 and FABP3 were both elevated in subjects with PAD and those with ACS. However, TnI was only elevated in ACS and, therefore, can act as a useful secondary biomarker to distinguish between PAD and ACS. FABP4 has been shown to be released from adipocytes while associating with lipolysis and possibly acting as an adipokine. Elevation of circulating FABP4 levels is associated with atherosclerosis, and cardiovascular events.

TABLE 5
Comparison of multiple biomarkers in non-PAD, PAD and ACS.

| | | | Acute coronary |
|---|---|---|---|
| | Non-PAD | PAD | syndrome (ACS) |
| Troponin pg/mL | 343 | 361 | 11.55 |
| FABP4 ng/mL | 8.61 | 21.85 | 38.50 |
| FABP3 ng/mL | 2.33 | 4.852 | 31.48 |
| ABI | 1.2 | 0.62 | 1.0 |

From Table 5, it can be seen that FABP3 or FABP4 can independently be used to diagnose patients with PAD, as levels are elevated compared to controls but not to the extent seen in ACS. As shown in table 5, there was a 2.5 fold increase in FABP4 levels in PAD patients relative to non-PAD patients. Troponin is a useful secondary marker to assist in ruling out ACS as the reason for the elevation. Combinations of all three of three protein markers, “FABP3 or FABP4” alone or in combination with troponin, increase the sensitivity and specificity for detection of PAD.

### Example 4: Large Study of PAD and Non-PAD Subjects

### Methodology

Our study cohort included 451 peripheral artery disease (PAD) and 188 non-PAD subjects. The PAD group was further categorized into asymptomatic, symptomatic and non-compressible (NC) ABI according to the clinical diagnosis established by an expert in PAD. Symptomatic patients were defined as patients with evidence of PAD on ultrasound who as well suffer from claudication. Asymptomatic patients were defined as patients with evidence of PAD on ultrasound who do not have a clinical symptoms of claudication. Patients with non-compressible (NC) ABI where defined as PAD patients with evidence of PAD on ultrasound and an abnormally elevated ABI above 1.3. A box-and-whisker plot was used to demonstrate the significant differences between PAD subgroups and non-PAD group in terms of FABP3 (ng/ml) plasma concentration levels. In this context, mean FABP3 values were calculated to be compared between groups using the Mann-Whitney U test.

Furthermore, subgroup analysis was conducted between non-PAD and PAD groups. In this subgroup analysis, confounding factors, namely, sex, hypertension, hypercholesteremia, diabetes, smoking, age, and coronary arterial disease (CAD), were utilized to measure the effect of either their presence or absence in our study groups. Once again, mean FABP3 values were calculated to be compared between groups using the Mann-Whitney U test.

Propensity score matching algorithm was performed using the full study cohort (451 PAD and 188 non-PAD subjects), to reduce confounders effect, improve the homogeneity of the case mix and eventually identify comparable groups for analysis. A logistic regression model was conducted to estimate the propensity score for each subject. Our list of potential confounding variables used in the model included age, sex, hypertension, hypercholesteremia, diabetes, smoking and CAD. The demographics and clinical characteristics matched cohorts (80 PAD and 80 non-PAD subjects) were expressed as means with standard deviations or percentages in a separate table and were compared using Mann-Whitney U test for continuous variables and chi-square test for categorical variables. Also, for the matched cohorts, correlation between FABP3 (ng/ml) plasma concentration levels and Ankle-Brachial Index (ABI) was analyzed using Spearman's correlation and correlation coefficient was calculated.

On the other hand, for the PAD group, walking distance was plotted against FABP3 (ng/ml) plasma concentration levels in an illustration to visualize the degree of linear relationship between both factors. A linear regression was conducted to statistically measure the magnitude of this association.

Finally, a group of 69 PAD patients where followed over a period of 12 months. At 0 and 12 months, blood samples were collected and ABI were measured for each patient. Using this data, cox proportional hazards regression was used to estimate the association of FABP3 with 15% change in ABI. This percentage difference in ABI value was identified based on our experience and understanding of a meaningful clinical change in ABI. FABP3 was modelled continuously per one standard deviation of FABP3. Follow-up continued up to 12 months after initial assessment, and individuals were censored for death or loss to follow-up. A sequence of Cox models was evaluated. After an unadjusted model, we adjusted for age, CAD, diabetes and smoking. All analyses were carried out at a 5% two-sided significance level and carried out using SPSS software version 23 (SPSS Inc., Chicago, Ill., USA).

### Results

FIG. 9 shows that, relative to non-PAD patients, the levels of FABP3 were statistically elevated in PAD (Symptomatic and Asymptomatic patients). We also noted an increase in FABP3 in PAD patients with non-compressible ABI. This data shows the benefits of FABP3 in distinguishing PAD patients from non-PAD patients regardless of the presence PAD symptoms. Also, FABP3 can be used to identify PAD patients from non-PAD patients when the ABI is non-compressible. FABP3 overcomes the failure of ABI in discriminating PAD from non-PAD.

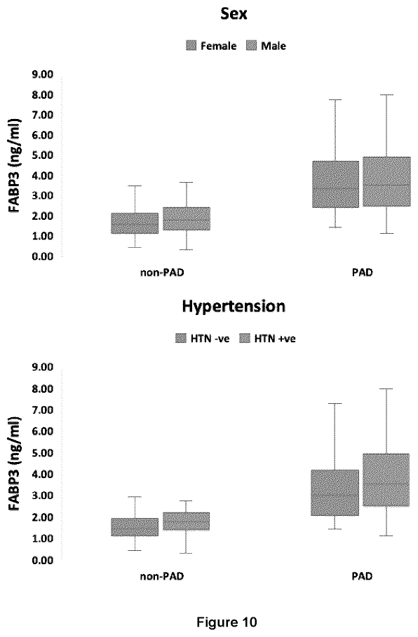

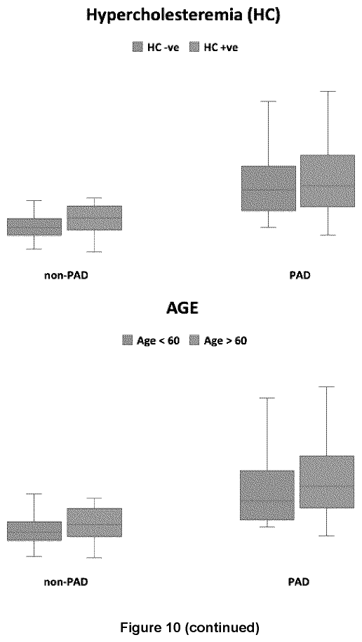

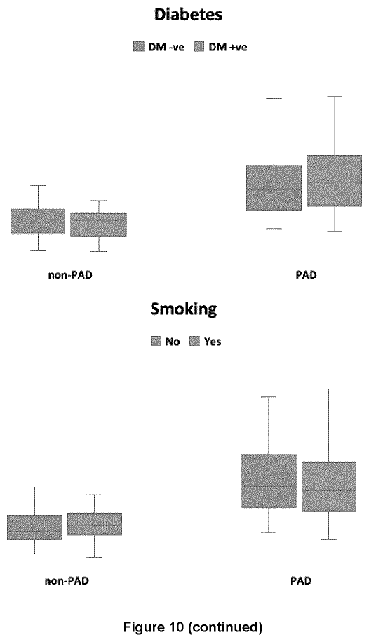

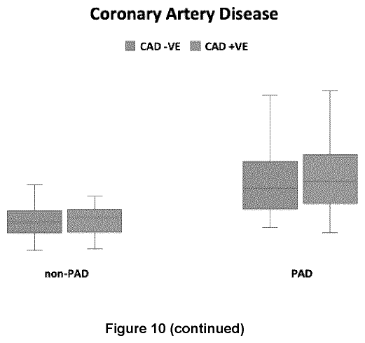

In order to better understand the relationship between the levels of FABP3 in PAD, we conducted a subgroup analysis looking at potential confounding factors (age <60, sex; hypertension; hypercholesteremia; diabetes; smoking; coronary artery disease). Here, levels of FABP3 were compared among patients with/without confounding factor against patients with/without PAD (FIG. 10). Our analysis shows that PAD patients have higher levels of FABP3 than non-PAD patients, irrespective of age <60, sex; hypertension; hypercholesteremia; diabetes; smoking; coronary artery disease.

We matched 80 non-PAD to 80 PAD patient based on age, sex, Hypertension, Hypercholesteremia, Diabetes, Smoking, CAD. As expected, there were no significant differences between non-PAD and PAD groups in the measured risk factors except for ABI. Relative to non-PAD patients, FABP3 levels were significantly higher in PAD patients (PAD 3.56 ng/mL, non-PAD 2.2 ng/mL, P<0.001; Table 6). This data demonstrates that after accounting for confounding factors, FABP3 was still elevated in PAD. This information further shows that FABP3 is elevated due to PAD and not a confounding cardiovascular risk factor such as age, sex, Hypertension, Hypercholesteremia, Diabetes, Smoking, CAD.

TABLE 6
Propensity score matching among PAD and non-PAD patients based on
Age <60, sex, Hypertension, Hypercholesteremia, Diabetes, Smoking,
CAD. Along with ABI, FBAP3 was the only variable that is statistically
different among the matched PAD and non-PAD patients.

| | | |
|---|---|---|
| Non-PAD | PAD | |
| (n= 80) | (n = 80) | p-value |

Mean (SD)

| | | | | | |
|---|---|---|---|---|---|
| FABP3 (ng/ml) | 2.20 | (0.84) | 3.56 | (0.53) | 0.001 |
| ABI | 1.10 | (0.10) | 0.62 | (0.17) | 0.001 |
| Age | 65.5 | (11.6) | 64.8 | (11.1) | 0.676 |

Frequency (%)

| | | | | | |
|---|---|---|---|---|---|
| Sex, male | 52 | (65) | 52 | (65) | 1.00 |
| Hypertension | 55 | (69) | 55 | (69) | 1.00 |
| Hypercholesteremia | 52 | (65) | 48 | (60) | 0.624 |
| Diabetes | 19 | (24) | 26 | (33) | 0.291 |
| Smoking | 59 | (74) | 60 | (75) | 1.00 |
| CHF | 0 | (0) | 2 | (3) | 0.245 |
| CAD | 19 | (24) | 23 | (29) | 0.590 |
| Stroke | 4 | (5) | 7 | (9) | 0.240 |
| Statins | 41 | (59) | 52 | (69) | 0.227 |

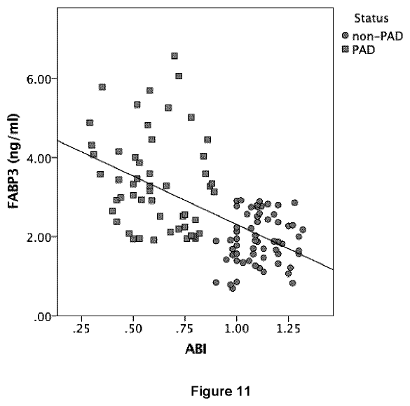

To better understand the association between FABP3 and PAD, we studied the hemodynamic correlation between FABP3 and ABI. Relative to the non-PAD patients, plasma FABP3 levels of PAD subjects were inversely correlated with ABI (r=−0.55, p-value<0.001; FIG. 11). This demonstrates that as PAD status worsens hemodynamically (as measured by ABI) within patients, circulating levels of FABP3 increase.

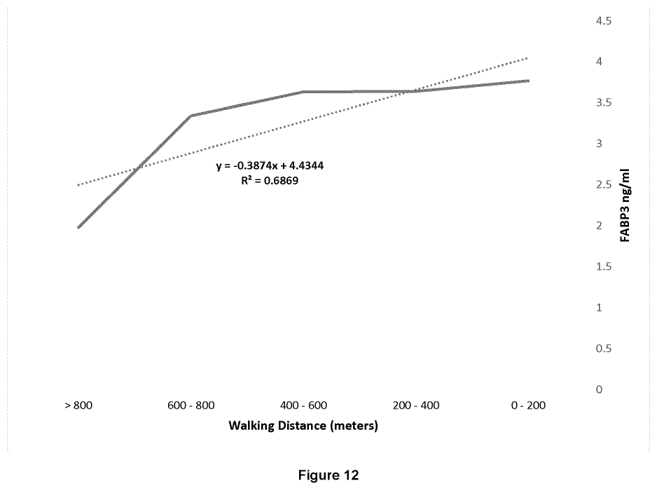

In FIG. 12, we studied the clinical correlation between FABP3 and ABI to better understand the association between FABP3 and PAD. Relative to the non-PAD patients, plasma FABP3 levels of PAD subjects were inversely correlated with walking distance (Correlation coefficient—0.39; p-value=0.001). This demonstrates that as walking distance decreases “increase in PAD severity” the circulating levels of FABP3 increases. Along with the ABI, this data added another layer of evidence on how worse clinical symptoms of PAD are related to increased levels of FABP3. This data strengths the association between PAD disease status and FABP3.

Finally, a group of 69 PAD patients where followed over a period of 12 months. FABP3 and ABI levels were compared at baseline to 12 months. This data allowed us to calculate the hazard ratio which estimates the association of FABP3 with 15% change in ABI. Our data showed a significant difference in progression of PAD “based on 15% change in ABI” and increased levels of FABP3 (Table 7). Based on this data, FABP3 is shown to predict 15% change in ABI (HR 1.28, 95% CI 1.06-1.65 per=one standard deviation of FABP3 difference. This model was improved after adjusting for confounding risk factors. Therefore, FABP3 can be used for prognostication of PAD disease status.

TABLE 7
Hazard ratios (95% confidence interval) for 15% change in
ABI per one standard deviation of FABP3 difference. (n = 69)

| | HR | |
|---|---|---|
| | (95% CI) | p-value |
| Model 1 (unadjusted model) | 1.28 (1.06-1.65) | 0.04 |
| Model 1 + Age | 1.29 (1.06-1.66) | 0.04 |
| Model 1 + Age + CAD | 1.34 (1.09-1.73) | 0.02 |
| Model 1 + Age + CAD + Diabetes | 1.31 (1.10-1.70) | 0.04 |
| Model 1 + Age + CAD + Diabetes + Smoking | 1.32 (1.12-1.69) | 0.03 |

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1-106. (canceled)

107. Fatty acid-binding protein 3 (FABP3) and/or fatty acid-binding protein 4 (FABP4), optionally in combination with at least one other biomarker, for any one or more of: diagnosing peripheral artery disease (PAD), staging PAD, assessing revascularization status in a subject afflicted with PAD, distinguishing PAD patients from non-PAD patients regardless of the presence of PAD symptoms, distinguishing PAD patients with a non-compressible ABI from non-PAD patients, or for determining prognosis in PAD.

108. FABP3 and/or FABP4 of claim 107, wherein the at least one other biomarker comprises high sensitivity troponin, troponin I (TnI), troponin T (TnT), FABP3, FABP4, creatinine, or a combination thereof.

109. A panel of biomarkers for assessing peripheral artery disease (PAD), the panel comprising at least two biomarkers, including FABP3 and/or FABP4 and optionally at least one additional biomarker, wherein the at least one additional biomarker comprises creatinine and/or a biomarker associated with PAD or myocardial ischemia, for example, high sensitivity troponin, troponin I (TnI), troponin T (TnT), FABP3, FABP4, or a combination thereof.

110. The FABP3 and/or FABP4 of claim 107, wherein:
a detected level of FABP3 protein in a patient sample of <0.6 ng/ml, <0.7 ng/ml, <0.8 ng/ml, <0.9 ng/ml, <1.0 ng/ml, <1.1 ng/ml, <1.2 ng/ml, <1.3 ng/ml, <1.4 ng/ml, <1.5 ng/ml, <1.6 ng/ml, <1.7 ng/ml, <1.8 ng/ml, <1.9 ng/ml, <2.0 ng/ml, <2.1 ng/ml, or <2.2 ng/ml is suggestive that the subject is highly unlikely to have PAD; and/or
a detected level of FABP3 protein in a patient sample of ≥0.6 ng/ml and <4.5 ng/ml, such as ≥0.6 ng/ml, ≥0.7 ng/ml, ≥0.8 ng/ml, ≥0.9 ng/ml, ≥1.0 ng/ml, ≥1.1 ng/ml, ≥1.2 ng/ml, ≥1.3 ng/ml, ≥1.4 ng/ml, ≥1.5 ng/ml, ≥1.6 ng/ml, ≥1.7 ng/ml, ≥1.8 ng/ml, ≥1.9 ng/ml, ≥2.0 ng/ml, ≥2.1 ng/ml, or ≥2.2 ng/ml, and <3.5 ng/ml, <3.6 ng/ml, <3.7 ng/ml, <3.8 ng/ml, <3.9 ng/ml, <4.0 ng/ml, <4.1 ng/ml, <4.2 ng/ml, <4.3 ng/ml, <4.4 ng/ml, and <4.5 ng/ml is suggestive that the subject is at moderate risk of having PAD; and/or
a detected level of FABP3 protein in a patient sample of ≥3.5 ng/ml and ≤5.3 ng/ml, such as ≥3.5 ng/ml, ≥3.6 ng/ml, ≥3.7 ng/ml, ≥3.8 ng/ml, ≥3.9 ng/ml, ≥4.0 ng/ml, ≥4.1 ng/ml, ≥4.2 ng/ml, ≥4.3 ng/ml, ≥4.4 ng/ml, or ≥4.3 ng/ml, and <4.4 ng/ml, <4.5 ng/ml, <4.6 ng/ml, <4.7 ng/ml, <4.8 ng/ml, <4.9 ng/ml, <5.0 ng/ml, <5.1 ng/ml, <5.2 ng/ml, and <5.3 ng/ml is suggestive that the subject is at moderate-high risk of having PAD; and/or
a detected level of FABP3 protein in a patient sample of ≥4.6 ng/ml, ≥4.7 ng/ml, ≥4.8 ng/ml, ≥4.9 ng/ml, ≥5.0 ng/ml, ≥5.1 ng/ml, ≥5.2 ng/ml, or ≥5.3 ng/ml is suggestive that the subject is at high risk of having PAD; and/or
a detected level of FABP4 protein in a patient sample of <15 ng/ml, <16 ng/ml, <17 ng/ml, <18 ng/ml, <19 ng/ml, <20 ng/ml, <21 ng/ml, <22 ng/ml, <23 ng/ml, <24 ng/ml, or <25 ng/ml is suggestive that the subject has PAD.

111. An assay, for example a point-of-care assay, comprising the FABP3 and/or FABP4 of claim 1 or the panel of claim 109.

112. A kit comprising the FABP3 and/or FABP4 of claim 1 or the panel of claim 3 or the assay of claim 111.

113. A method for diagnosing or staging peripheral artery disease (PAD), such as non-symptomatic (stage 0), mild (stage 1), moderate (stage 2), severe (stage 3), early chronically threatened limb ischemia (CTLI)(stage 4) or advanced CTLI (stage 5 or 6) PAD in a subject, for example a subject free of clinical and/or biochemical evidence of myocardial ischemia or a subject free of clinical and/or biochemical evidence of kidney dysfunction or a subject free of clinical and/or biochemical evidence of acute stroke and/or acute muscle toxicity, the method comprising detecting the level of fatty acid-binding protein 3 (FABP3) and/or FABP4 in the subject; wherein an elevated level of FABP3 and/or FABP4 is indicative of PAD in the subject;
wherein optionally the elevated level of FABP3 and/or FABP4 in the subject is determined by comparing the detected level of FABP3 and/or FABP4 to a control level of FABP3 and/or FABP4, such as a predetermined value obtained from one or a pool of non-PAD patients or healthy patients.

114. The method of claim 113, wherein the method further comprises detecting the level of at least one additional biomarker, wherein the at least one additional biomarker optionally comprises the other of FABP3 and/or FABP4, high sensitivity troponin, TnI, TnT, and/or creatinine, wherein a substantially normal level of high sensitivity troponin, TnI, TnT and/or creatinine in the subject is further indicative of PAD in the subject.

115. The method claim 113, wherein the subject has a concurrent condition, optionally selected from the group consisting of kidney dysfunction, stroke, diabetes, and/or muscle toxicity and optionally wherein the detected level of FABP3 and/or FABP4 and/or the control level of FABP3 and/or FABP4 is adjusted for the concurrent condition.

116. The method of claim 113, wherein the method further comprises assessing the ABI of the subject.

117. A method for assessing revascularization in a subject with peripheral artery disease (PAD), including asymptomatic PAD (stage 0), mild PAD (stage 1), moderate PAD (stage 2), severe PAD (stage 3), early CTLI (stage 4) or advanced CTLI (stages 5-6), for example a subject free of clinical and/or biochemical evidence of myocardial ischemia and/or free of clinical and/or biochemical evidence of kidney dysfunction and/or free of clinical and/or biochemical evidence of acute stroke and/or muscle toxicity,
the method comprising detecting the level of fatty acid-binding protein 3 (FABP3) and/or FABP4 in the subject,
and optionally detecting the level of at least one additional biomarker, for example, the other of FABP3 and/or FABP4, high sensitivity troponin, TnI, TnT, and/or creatinine;
wherein a substantially normal level of FABP3 and/or FABP4 or a reduction in an elevated level of FABP3 and/or FABP4 is indicative of arterial revascularization in the subject, and wherein, optionally:
the substantially normal level of FABP3 and/or FABP4 or the reduction in the elevated level of FABP3 and/or FABP4 is determined by comparing the detected level of FABP3 and/or FABP4 to a control level of FABP3 and/or FABP4; or wherein the control level of FABP3 and/or FABP4 is a predetermined value obtained from one or a pool of non-PAD patients or healthy patients; or wherein the control level of FABP3 and/or FABP4 is a predetermined value obtained from one or a pool of PAD patients; or wherein the control level of FABP3 and/or FABP4 is the level of FABP3 and/or FABP4 detected in the subject prior to revascularization treatment.

118. The method of claim 117, wherein the method further comprises detecting the level of high sensitivity troponin, troponin I (TnI), troponin T (TnT) and/or creatinine in the subject, wherein a substantially normal level of high sensitivity troponin, TnI, TnT and/or creatinine in the subject is further indicative of revascularization in the subject,
and optionally the substantially normal level of high sensitivity troponin, TnI, TnT and/or creatinine in the subject is determined by comparing the detected level of high sensitivity troponin, TnI, TnT and/or creatinine to a control level of high sensitivity troponin, TnI, Tnt, and/or creatinine.

119. The method of claim 117, wherein the subject has a concurrent condition such as kidney dysfunction, stroke, diabetes, and/or muscle toxicity and wherein the detected level of FABP3 and/or FABP4 and/or the control level of FABP3 and/or FABP4 is optionally adjusted for the concurrent condition.

120. A method for predicting whether a subject with peripheral artery disease (PAD) is likely to progress to CTLI, the method comprising detecting the level of fatty acid-binding protein 3 (FABP3) and/or FABP4 in the subject; wherein the extent of elevation of FABP3 and/or FABP4 is correlated with the likelihood of the subject progressing to CTLI,
wherein optionally the extent of elevation of FABP3 and/or FABP4 in the subject is determined by comparing the detected level of FABP3 and/or FABP4 to a control level of FABP3 and/or FABP4, or the control level of FABP3 and/or FABP4 is a predetermined value obtained from one or a pool of non-PAD patients or healthy patients;
wherein optionally the subject is free of clinical and/or biochemical evidence of myocardial ischemia or free of clinical and/or biochemical evidence of kidney dysfunction or free of clinical and/or biochemical evidence of acute stroke and/or muscle toxicity.

121. The method of claim 120, wherein the method further comprises detecting the level of at least one additional biomarker, for example, the other of FABP3 and/or FABP4, high sensitivity troponin, TnI, TnT, and/or creatinine; wherein, where the method further comprises detecting the level of troponin, TnI, TnT or creatinine, a substantially normal level of high sensitivity troponin, TnI creatinine and/or TnT in the subject is further indicative of PAD in the subject; further optionally comprising assessing the ABI of the subject.

122. The method of claim 120, wherein the subject has a concurrent condition such as kidney dysfunction, stroke, diabetes and/or muscle toxicity, and wherein the detected level of FABP3 and/or FABP4 and/or the control level of FABP3 and/or FABP4 is optionally adjusted for the concurrent condition.

123. The method of claim 120, the method further comprising treating the subject based upon the outcome of the method.

124. A method of treating a subject with peripheral artery disease, the method comprising carrying out the method of claim 120 and treating the subject based upon the outcome of the method.

125. Use of FABP3 and/or FABP4 for diagnosing peripheral artery disease (PAD) in a subject, wherein an elevated level of FABP3 and/or FABP4 is indicative of PAD in the subject.

126. Use of FABP3 and/or FABP4 for assessing revascularization in a subject with peripheral artery disease (PAD); wherein a substantially normal level of FABP3 and/or FABP4 or a reduction in an elevated level of FABP3 is indicative of arterial revascularization in the subject.

127. Use of FABP3 and/or FABP4 for predicting whether a subject with peripheral artery disease (PAD) is likely to progress to CTLI, wherein the extent of elevation of FABP3 and/or FABP4 is correlated with the likelihood of the subject progressing to CTLI.
